(19) United States
(12) Patent Application Publication
(10) Pub. No.: US 2018/0312592 A1
(43) Pub. Date: Nov. 1, 2018

(54) CYSTEINE-SUBSTITUTED IMMUNOGLOBULINS

(71) Applicant: CELLERANT THERAPEUTICS, INC., San Carlos, CA (US)

(72) Inventors: Jagath R. JUNUTULA, Fremont, CA (US); Ying-Ping JIANG, Lafayette, CA (US); Jianqing HUANG, Foster City, CA (US); Madhavi MISHRA, Fremont, CA (US)

(21) Appl. No.: 15/745,351

(22) PCT Filed: Jul. 15, 2016
(86) PCT No.: PCT/US2016/042645

§ 371 Date: Jan. 16, 2018

Related U.S. Application Data
(60) Provisional application No. 62/193,531, filed on Jul. 16, 2015.

(51) Int. Cl. C07K 16/28; A61K 47/68; G01N 33/574; A61P 35/00

(52) U.S. Cl. CPC C07K 16/2851; A61K 47/6849; G01N 33/574; A61P 35/00; A61K 2039/505; C07K 2317/52; C07K 2317/21; C07K 2317/56; C07K 2317/565; A61K 47/6803

(57) ABSTRACT

The disclosure provides cysteine substituted immunoglobulins, including polypeptides, antibodies, nucleic acids encoding such polypeptides and antibodies, host cells, vectors and processes for making the same, conjugated derivatives of the antibodies, compositions and methods of making such antibodies and conjugated derivatives, and methods of using the antibodies and conjugated variants for the detection and treatment of cancer and for killing diseased cells. In certain embodiments, the substitution is selected from V266C, G316C, H285C, R301C, V303C, T307C, Y436C and L441C (EU Numbering) or S156 in the hevy chain (under Kabat numbering).

### CROSS-REFERENCE TO RELATED PATENT APPLICATIONS

The present patent application is a continuation of Patent Cooperation Treaty Appl. No. PCT/US2016/042645, filed Jul. 15, 2016, which claims benefit of priority to U.S. Provisional Patent Application No. 62/193,531, filed Jul. 16, 2015, which is incorporated by reference for all purposes.

### REFERENCE TO SUBMISSION OF A SEQUENCE LISTING AS A TEXT FILE

The Sequence Listing written in file 1014170_ST25.txt created on Jul. 15, 2016, 10,366 bytes, machine format IBM-PC, MS-Windows operating system, is hereby incorporated by reference in its entirety for all purposes.

### BACKGROUND OF THE INVENTION

Monoclonal antibodies (mAbs) are an essential tool in research and therapy due to their high specificity and affinity for target antigens. Since the 1990's, therapeutic mAbs have made a substantial impact on medical care for a wide range of diseases, including inflammatory disorders and cancer. A critical feature of mAbs is their ability to bind target antigens in a highly specific manner, marking them for removal by the host immune clearance methods, such as complement-dependent cytotoxicity (CDC) or antibody-dependent cell-mediated cytotoxicity (ADCC). Antibodies can also impart therapeutic benefit by binding an inhibiting the function of target antigens, as in the case of trastuzumab (Herceptin®), bevacizumab (Avastin®) and cetuximab (Erbitux®).

CLL-1 is a cell surface glycoprotein predominantly expressed in myeloid cells found in hematologic malignancies, such as leukemias (e.g., acute myeloid leukemia (AML)). The currently available therapies for hematologic malignancies carry adverse and often severe side effects. For example, complications arising from MYLOTARG® administration include hepatotoxicity, veno-occlusive disease, severe myelosuppression (in −98% of patients), tumor lysis syndrome, immune hypersensitivity syndrome and respiratory disorders. Thus, there is a need to identify new therapies for hematologic malignancies that are efficacious with reduced side effects. Since CLL-1 is selectively expressed on myeloid cells, compositions that recognize and bind to CLL-1 may be useful for such therapies of hematologic malignancies, especially those of myeloid origin.

Conjugation to cytotoxic drugs or radionuclides can expand the utility of mAbs and improve their potency and effectiveness. This is accomplished because the antibody targets and delivers the cytotoxic payload specifically to the diseased tissue. Antibodies have been conjugated to a number of cytotoxic drugs, through various linker chemistries and these antibody drug conjugates (ADCs) have the ability to selectively and potently kill antigen-expressing tumor cells. ADCs have demonstrated success in the clinic, and there are two such drugs, ado-trastuzumab emtansine (Kadcyla®) and brentuximab vendotin (Adcetris®) are commercially available.

The successful development of an ADC depends upon the optimization of antibody selection, linker stability, cytotoxic drug potency and mode of linker-drug conjugation to the antibody.

In a conventional ADC, drug conjugation yields heterogeneous products, containing a mixture of species with different molar ratios of drug to antibody. The conjugation site to the antibody occurs at solvent accessible, reactive amino acid residues such as lysines or cysteines. The heterogeneity occurs as two levels, in that each ADC species differs in both drug load and conjugation site. Panowski et al., mAbs 6:1, 31-45 (2014). Therefore, each species may have distinct properties, resulting in a wide range of in vivo pharmacokinetic (PK) properties as well as batch-to-batch variability. Additionally, the variable drug-to-antibody ratio (DAR) results in a high drug load, high hydrophobicity, fast clearance, lower tolerability and a narrow therapeutic window. Junutula et al., Nat. Biotech. 26(8), 925-932 (2008).

Site-specific conjugation, in which a known number of linker-drugs are consistently conjugated to defined sites, is one way to overcome these challenges. Heterogeneity is minimized and ADC properties are more predictable, with consistent inter-batch conjugate production.

The amino acid cysteine provides a reactive thiol group. This group has long been used as the location to label proteins, as well as for the generation of ADCs. While cysteines can be engineered into proteins, this approach is not without challenges. For example, the engineered free cysteine can conjugate with cysteines on other molecules to form protein-dimers. It can also pair intra-molecularly with native cysteine residues to create improper folding to impair or inhibit protein function. Hence the success of using introduced cysteine residues for site-specific conjugation relies on the ability of select proper sites in which cysteine-introduced substitution does not alter antibody structure or function. Junutula et al., Nat. Biotech. 30(2): 184-191 (2012).

A further complexity is that solvent accessibility and charge at a substitution site is important for ADC stability. In a study of stability of cysteine, engineered anti-Her2/neu maleimide linker ADCs, high solvent accessibility lost conjugated thiol-reactive linkers in plasma as a result of maleimide exchange with reactive thiols in albumin, free cysteine or glutathione. Shen et al., Nat. Biotech. 30(2): 184-191 (2012). Hence, there is still a great need to identify stable, site-specific ADCs which have consistent drug load, low hydrophobicity, slow clearance, high tolerability and a greater therapeutic index. Furthermore, there is an even greater need to create stable, site-specific ADCs that target CLL-1.

The statements in this Background are not neither admissions of prior art nor endorsements of the cited references.

### BRIEF SUMMARY OF THE INVENTION

The disclosure provides cysteine substituted immunoglobulins, including polypeptides, antibodies, nucleic acids encoding such polypeptides and antibodies, host cells, vectors and processes for making the same, conjugated derivatives of the antibodies, compositions and methods of making such antibodies and conjugated derivatives, and methods of using the antibodies and conjugated variants for the detection and treatment of cancer and for killing diseased cells.

In one embodiment, this disclosure provides a cysteine substituted immunoglobulin polypeptide, wherein the substituted residue is one or more residues selected from the group consisting of: V266C, H285C, R301C, V303C, T307C, G316C, Y436C and L441C (EU numbering). In one aspect, the immunoglobulin polypeptides are derived from human IgG heavy chain constant regions. In another aspect, the IgG is isotype IgG1, IgG2, IgG3 or IgG4.

In another embodiment, the disclosure provides isolated nucleic acid sequences encoding a cysteine substituted immunoglobulin polypeptide, wherein the substituted residue is one or more residues selected from the group consisting of: V266C, H285C, R301C, V303C, T307C, G316C, Y436C and L441C (EU numbering). In one aspect, the nucleic acid is operably linked with an expression control sequence. In another aspect, the operably linked nucleic acid further comprised in an expression vector. In yet another aspect, the disclosure provides host cells comprising the expression vectors.

In yet another embodiment, the disclosure provides a process for making a cysteine-substituted immunoglobulin polypeptide comprising culturing a recombinant cell comprising a nucleic acid molecule further comprising a nucleotide sequence encoding a cysteine-substituted immunoglobulin polypeptide, wherein the substituted residue is one or more residues selected from the group consisting of: V266C, H285C, R301C, V303C, T307C, G316C, Y436C and L441C (EU numbering).

In a further embodiment, the disclosure provides cysteine substituted antibody comprising a cysteine-substituted immunoglobulin polypeptide further comprising a substituted amino acid residue selected from the group consisting of: V266C, H285C, R301C, V303C, T307C, G316C, Y436C and L441C (EU numbering) in a heavy chain constant region. In one aspect the heavy chain constant region is derived from a human IgG isotype selected from the group consisting of IgG1, IgG2, IgG3 and IgG4.

In another aspect, the antibody further comprises a light chain. In a further aspect, the light chain is selected from the group consisting of kappa and lambda.

In yet another aspect the antibody binds to CLL-1, GPR114, IL1RAP, TIM-3, CD19, CD20, CD22, ROR1, mesothelin, CD33, CD123/IL3Ra, c-Met, PSMA, prostatic acid phosphatase (PAP), CEA, CA-125, Muc-1, AFP, Glycolipid F77, EGFRvIII, GD-2, NY-ESO-1 TCR, tyrosinase, TRPI/gp75, gp100/pmel-17, Melan-A/MART-1, Her2/neu, WT1, EphA3, telomerase, HPV E6, HPV E7, EBNA1, BAGE, GAGE and MAGE A3 TCRSLITRK6, ENPP3, Nectin-4, CD27, SLC44A4, CAIX, Cripto, CD30, MUC16, GPNMB, BCMA, Trop-2, Tissue Factor (TF), CanAg, EGFR, αv-integrin, CD37, Folate Receptor, CD138, CEACAM5, CD56, CD70, CD74, GCC, 5T4, CD79b, Steap1, Napi2b, Lewis Y Antigen, LIV, c-RET, DLL3,EFNA4, or Endosialin/CD248. In a further aspect, the antibody binds to CLL-1 and comprises a variable light chain and a variable heavy chain, wherein:
(a) the variable light chain further comprises a CDR-L1, CDR-L2 and CDR-L3, further wherein:

(SEQ ID NO: 1)
a. CDR-L1 is ESVDSYGNSF
(SEQ ID NO: 2)
b. CDR-L2 is LAS
(SEQ ID NO: 3)
c. CDR-L3 is QQNNYDPWT,
and

(b) the variable heavy chain further comprising a CDR-H1, CDR-H2 and CDR-H3, further wherein:

(SEQ ID NO: 4)
a. CDR-H1 is GYTFTSYV
(SEQ ID NO: 5)
b. CDR-H2 is INPYNDGT,
and
(SEQ ID NO: 6)
c. CDR-H3 is ARPIYFDNDYFDY.

In a further aspect, the antibody binds to CLL-1 and comprises a variable light chain and a variable heavy chain, wherein:
(c) the variable light chain further comprises a CDR-L1, CDR-L2 and CDR-L3, further wherein:

(SEQ ID NO: 13)
a. CDR-L1 is RATQELSGYLS
(SEQ ID NO: 14)
b. CDR-L2 is AASTLDS
(SEQ ID NO: 15)
c. CDR-L3 is LQYAIYPYT,
and

(d) the variable heavy chain further comprising a CDR-H1, CDR-H2 and CDR-H3, further wherein:

(SEQ ID NO: 16)
a. CDR-H1 is GYTFTSYFIH
(SEQ ID NO: 17)
b. CDR-H2 is FINPYNDGSK,
and
(SEQ ID NO: 18)
CDR-H3 is DDGYYGYAMDY

In some embodiments, the anti-CLL-1 antibody comprises a light chain variable region sequence comprises DIQMTQSPSSLSASVGDRVTLTCRATQELSGYLSWLQQKPGKAIKRLIYAASTLDSGV PSRFSGNRAGTDYTLTISSLQPEDFATYYCLQYAIYPYTFGQGTKLEIK (SEQ ID NO:19), a heavy chain variable region sequence comprises EVQLVQSGAEVKKPGASVKMSCKASGYTFTSYFIHWVRQAPGQGLEWIGFINPYND GSKYAQKFQGRATLTSDKSTSTVYMELSSLRSEDTAVYYCTRDDGYYGYAMDYWG QGTLVTVSS (SEQ ID NO:20), or both of the light and heavy chain sequences above.

In a further aspect, the disclosure provides isolated nucleic acid sequences encoding a cysteine substituted antibody. In one aspect, the nucleic acid is operably linked with an expression control sequence. In another aspect, the operably linked nucleic acid further comprises an expression vector. In a further aspect, the disclosure provides host cells comprising the expression vectors and methods of making antibodies comprising culturing such host cells. In a further aspect, the disclosure provides isolating the antibody.

In yet another embodiment, the disclosure provides a cysteine substituted antibody, wherein the substituted cysteine is connected through a linker to a conjugated moiety. In one aspect, the conjugated moiety is selected from the group consisting of: drug, radionucleotide, fluorophore, biotin, RNA, antibiotic, protein and a detectable moiety.

In another aspect, the conjugated moiety is a drug, biotin (BMCC or HPDP) or fluorophore (Alexa488). In yet another aspect, the drug is selected from the group consisting of: a benzodiazepine derivative (including but not limited to a pyrrolo benzodiazepine, an indolino benzodiazepine or an isoquinolidino benzodiazepine), which can be in monomer or dimer form (e.g., a heterodimer or homodimer, such as pyrrolobenzodiazepine (PBD) dimer, indolinobenzodiazepine dimer, isoquinolidinobenzodiazepine dimer (including but not limited to D202 as described below), a dolastatin, an auristatin, maytansinoid, tubulysin, cryptophycin, alpha-amanitin, trichothene, SN-38, duocarmycin, CC1065, calicheamincin, an enediyne antibioatic, taxane, doxorubicin derivatives, anthracycline and stereoisomers, azanofide, isosteres, analogs or derivatives thereof.

In a further aspect, the linker is covalently bonded to the drug. In another aspect, the linker is attached to the antibody through a reaction between a thiol and a thiol reactive group, e.g., maleimide, halide and sulfonyl. In yet another aspect, the linker is connected via a disulfide bond to the drug. In a further aspect, the disulfide bond is a pyridyl disulfide moiety. In a further aspect, the linker is cleavable in the microenvironment of the target.

In a further aspect, the conjugated moiety is a detectable moiety. In a further aspect the detectable moiety is a fluorophore such as A488 or a biotin (e.g., BMCC-biotin or HPDP-biotin).

In a further embodiment, the disclosure provides compositions comprising the cysteine substituted antibodies and an adjuvant. In one aspect the adjuvant is pharmaceutically acceptable carrier or diluent.

In a further embodiment, the disclosure provides a method of detecting the presence of a cell of interest, comprising contacting a cell with at least an effective amount of a cysteine-substituted antibody capable of binding the cell, and detecting binding of the antibody to the cell, wherein said binding indicates the cell of interest. In one aspect, the cell of interest is a cell expressing CLL-1. In another aspect, the cysteine-substituted antibody is conjugated to a detectable moiety.

In a further embodiment, the disclosure provides a method of diagnosing a disease comprising: (i) contacting a biological sample from an individual with at least an effective amount of a cysteine substituted antibody capable of binding to diseased cells, and (ii) detecting binding of the antibody to a diseased cell, wherein binding indicated the presence of the disease. In one aspect, the substituted cysteine antibody (CYSMAB) is conjugated to a detectable moiety. In another aspect, the disease is cancer, and the antibody bonding to a tumor associated antigen or a cancer stem cell associated antigen. In yet another aspect, the disease is a myeloproliferative disorder. In a further aspect, the myeloproliferative disorder is selected form the group consisting of AML, CML, CMML, multiple myeloma, plasmacytoma and myelofibrosis. In a further aspect, the tumor associated antigen or cancer stem cell antigen is CLL-1, GPR114, IL1RAP, TIM-3, CD19, CD20, CD22, ROR1, mesothelin, CD33, CD123/IL3Ra, c-Met, PSMA, prostatic acid phosphatase (PAP), CEA, CA-125, Muc-1, AFP, Glycolipid F77, EGFRvIII, GD-2, NY-ESO-1 TCR, tyrosinase, TRPI/gp75, gp100/pmel-17, Melan-A/MART-1, Her2/neu, WT1, EphA3, telomerase, HPV E6, HPV E7, EBNA1, BAGE, GAGE and MAGE A3 TCRSLITRK6, ENPP3, Nectin-4, CD27, SLC44A4, CAIX, Cripto, CD30, MUC16, GPNMB, BCMA, Trop-2, Tissue Factor (TF), CanAg, EGFR, αv-integrin, CD37, Folate Receptor, CD138, CEACAM5, CD56, CD70, CD74, GCC, 5T4, CD79b, Steap1, Napi2b, Lewis Y Antigen, LIV, c-RET, DLL3,EFNA4, or Endosialin/CD248.

In a further embodiment, the disclosure provides a method of inhibiting cell division comprising contacting a cell with at least an effective amount of a cysteine substituted conjugate (CYSMAB) capable of binding to the cell and which is conjugated to a drug that is cytotoxic to the cell. In one aspect, inhibition of cell division results in cell death. In another aspect, the cell is a tumor or cancer stem cell and the antibody binds to a tumor associated antigen or cancer stem cell antigen. In another aspect, the tumor or cancer stem cells are from a myeloproliferative disorder. In yet another aspect, the myeloproliferative disorder is selected from the group consisting of AML, CML, CMML, multiple myeloma, plasmacytoma and myelofibrosis. In a further aspect, the tumor associated antigen or cancer stem cell antigen is CLL-1, GPR114, IL1RAP, TIM-3, CD19, CD20, CD22, ROR1, mesothelin, CD33, CD123/IL3Ra, c-Met, PSMA, prostatic acid phosphatase (PAP), CEA, CA-125, Muc-1, AFP, Glycolipid F77, EGFRvIII, GD-2, NY-ESO-1 TCR, tyrosinase, TRPI/gp75, gp100/pmel-17, Melan-A/MART-1, Her2/neu, WT1, EphA3, telomerase, HPV E6, HPV E7, EBNA1, BAGE, GAGE and MAGE A3 TCRSLITRK6, ENPP3, Nectin-4, CD27, SLC44A4, CAIX, Cripto, CD30, MUC16, GPNMB, BCMA, Trop-2, Tissue Factor (TF), CanAg, EGFR, αv-integrin, CD37, Folate Receptor, CD138, CEACAM5, CD56, CD70, CD74, GCC, 5T4, CD79b, Steap1, Napi2b, Lewis Y Antigen, LIV, c-RET, DLL3,EFNA4, or Endosialin/CD248.

In a further embodiment, the disclosure provides a method of treating cancer comprising administering to a patient a therapeutically effective amount of a cysteine substituted antibody conjugate (e.g., an antibody-drug conjugate (ADC) generated using cysteine substituted antibody), wherein the antibody conjugate is capable of binding a tumor associated antigen or cancer stem cell antigen. In one aspect, the cancer is a myeloproliferative disorder. In another aspect, the myeloproliferative disorder is selected form the group consisting of AML, CML, CMML, multiple myeloma, plasmacytoma and myelofibrosis. In yet another further aspect, the tumor associated antigen or cancer stem cell antigen is CLL-1, GPR114, IL1RAP, TIM-3, CD19, CD20, CD22, ROR1, mesothelin, CD33, CD123/IL3Ra, c-Met, PSMA, prostatic acid phosphatase (PAP), CEA, CA-125, Muc-1, AFP, Glycolipid F77, EGFRvIII, GD-2, NY-ESO-1 TCR, tyrosinase, TRPI/gp75, gp100/pmel-17, Melan-A/MART-1, Her2/neu, WT1, EphA3, telomerase, HPV E6, HPV E7, EBNA1, BAGE, GAGE and MAGE A3 TCRSLITRK6, ENPP3, Nectin-4, CD27, SLC44A4, CAIX, Cripto, CD30, MUC16, GPNMB, BCMA, Trop-2, Tissue Factor (TF), CanAg, EGFR, αv-integrin, CD37, Folate Receptor, CD138, CEACAM5, CD56, CD70, CD74, GCC, 5T4, CD79b, Steap1, Napi2b, Lewis Y Antigen, LIV, c-RET, DLL3,EFNA4, or Endosialin/CD248.

Also provided is an antibody conjugate comprising a cysteine-substituted immunoglobulin polypeptide comprising a substituted amino acid residue at S156 under Kabat numbering (157 under EU numbering) in the antibody heavy chain (the antibody portion having a heavy chain and a light chain) linked via the cysteine to an indolinobenzodiazepine dimer or isoquinolidinobenzodiazepine dimer (including but not limited to D202 as described below). In some embodiments, the indolinobenzodiazepine dimer or isoquinolidinobenzodiazepine dimer (including but not limited to D202 as described below) is attached to the antibody through a linker and the linker is connected via disulfide bond to the drug. In some embodiments, the disulfide bond is a pyridyl disulfide moiety. In some embodiments, the linker is cleavable in the microenvironment of the target.

Also provided is a composition comprising an antibody conjugate comprising a cysteine-substituted immunoglobulin polypeptide comprising a substituted amino acid residue at S156 under Kabat numbering (157 under EU numbering) in the antibody heavy chain linked via the cysteine to an indolinobenzodiazepine dimer or isoquinolidinobenzodiazepine dimer (including but not limited to D202 as described below) and an adjuvant. In some embodiments, the composition is pharmaceutically acceptable.

Also provided is a method of inhibiting cell division comprising contacting a cell with at least an effective amount of an antibody conjugate comprising a cysteine-substituted immunoglobulin polypeptide comprising a substituted amino acid residue at S156 under Kabat numbering (157 under EU numbering) in the antibody heavy chain linked via the cysteine to an indolinobenzodiazepine dimer or isoquinolidinobenzodiazepine dimer (including but not limited to D202 as described below). In some embodiments, the inhibition of cell division results in cell death. In some embodiments, the cell is a tumor or cancer stem cell, and the antibody binds to a tumor associated antigen or cancer stem cell antigen. In some embodiments, the tumor or cancer stem cells are from a myeloproliferative disorder. In some embodiments, the myeloproliferative disorder is selected from the group consisting of: AML, CML, CMML, multiple myeloma, plamacytoma myelofibrosis. In some embodiments, the tumor associated antigen or cancer stem cell antigen is CLL-1, GPR114, IL1RAP, TIM-3, CD19, CD20, CD22, ROR1, mesothelin, CD33, CD123/IL3Ra, c-Met, PSMA, prostatic acid phosphatase (PAP), CEA, CA-125, Muc-1, AFP, Glycolipid F77, EGFRvIII, GD-2, NY-ESO-1 TCR, tyrosinase, TRPI/gp75, gp100/pmel-17, Melan-A/MART-1, Her2/neu, WT1, EphA3, telomerase, HPV E6, HPV E7, EBNA1, BAGE, GAGE and MAGE A3 TCRSLITRK6, ENPP3, Nectin-4, CD27, SLC44A4, CAIX, Cripto, CD30, MUC16, GPNMB, BCMA, Trop-2, Tissue Factor (TF), CanAg, EGFR, αv-integrin, CD37, Folate Receptor, CD138, CEACAM5, CD56, CD70, CD74, GCC, 5T4, CD79b, Steap1, Napi2b, Lewis Y Antigen, LIV, c-RET, DLL3, EFNA4, or Endosialin/CD248.

Also provided is a method of treating cancer comprising administering to a patient a therapeutically effective amount of an an antibody conjugate comprising a cysteine-substituted immunoglobulin polypeptide comprising a substituted amino acid residue at S156 under Kabat numbering (157 under EU numbering) in the antibody heavy chain linked via the cysteine to an indolinobenzodiazepine dimer or isoquinolidinobenzodiazepine dimer (including but not limited to D202 as described below) wherein the antibody conjugate is capable of binding a tumor associated antigen or cancer stem cell antigen. In some embodiments, the cancer is a myeloproliferative disorder. In some embodiments, the myeloproliferative disorder is selected from the group consisting of: AML, CML, CMML, multiple myeloma, plasmacytoma and myelofibrosis. In some embodiments, the tumor associated antigen or cancer stem cell antigen is CLL-1, GPR114, IL1RAP, TIM-3, CD19, CD20, CD22, ROR1, mesothelin, CD33, CD123/IL3Ra, c-Met, PSMA, prostatic acid phosphatase (PAP), CEA, CA-125, Muc-1, AFP, Glycolipid F77, EGFRvIII, GD-2, NY-ESO-1 TCR, tyrosinase, TRPI/gp75, gp100/pmel-17, Melan-A/MART-1, Her2/neu, WT1, EphA3, telomerase, HPV E6, HPV E7, EBNA1, BAGE, GAGE and MAGE A3 TCRSLITRK6, ENPP3, Nectin-4, CD27, SLC44A4, CAIX, Cripto, CD30, MUC16, GPNMB, BCMA, Trop-2, Tissue Factor (TF), CanAg, EGFR, αv-integrin, CD37, Folate Receptor, CD138, CEACAM5, CD56, CD70, CD74, GCC, 5T4, CD79b, Steap1, Napi2b, Lewis Y Antigen, LIV, c-RET, DLL3, EFNA4, or Endosialin/CD248.

Other objects of the disclosure may be apparent to one skilled in the art upon reading the following specification and claims.

### BRIEF DESCRIPTION OF THE DRAWINGS

FIG. 1 shows 3 distinct ELISA assay comparing the specificity of the antibody-fluorophore conjugates (AFCs) of the disclosure. Format 1 is a direct ELISA wherein the CLL-1 extra cellular domain (ECD) fixed, to which the AFC binds, and is then detected by the anti-fluorophore antibody (rabbit anti-A488) and detection reagent (anti-rabbit Fc-HRP). Format 2 is an ELISA wherein the anti-fluorophore antibody (anti-A488 Ab) is bound, and the AFC and biotinylated CLL-1 ECD are sandwiched between the detection reagent (SA-HRP). Format 3 is an alternative ELISA were anti-CLL-1 Ab is immobilized and sandwiches CLL-1 ECD and AFC and the anti-A488 Ab and the detection reagent (anti-rabbit Fc-HRP).

FIG. 2A-2B displays the assay ELISA specificity assay format 1. FIG. 2A shows similar specificity of the assay for labeled AFC and WT anti-fluorophore antibody, relative to IgG, trastuzumab, naked HuM31, and control labeled IgG. The results show similar specificity between labeled HuM31 and WT. FIG. 2B depicts the effect of interference with human plasma and PBS.

FIG. 3 is a cartoon showing the format of the stability ELISA assay. In this format, AFC is sandwiched between immobilized CLL-1 ECD and detection reagent. (SA-HRP).

FIGS. 4A-4E shows an alignment of the HuM31 heavy chain antibody constant chain with other IgG1, IgG2, IgG3 and IgG4 isotypes with residues identified with Kabat, EU Index and sequential numbering. Light Chain Sequences (FIG. 4A): M31 (SEQ ID NO:7), HuM31 (SEQ ID NO:8), kappa (SEQ ID NO:9) and lambda (SEQ ID NO:10). Heavy Chain Sequences (FIG. 4B): M31 (SEQ ID NO:11) and HuM31 (SEQ ID NO:12).

FIG. 5 illustrates fluorescence-to-antibody (FAR) ratios for various antibody conjugates.

FIG. 6 illustrates drug-to-antibody (DAR) ratios for various antibody conjugates.

FIG. 7 provides results of the conjugation, including amino acid residue and fluorophore-to-antibody ratio (“FAR”) for various antibody conjugates.

FIG. 8 provides results of the conjugation, including amino acid residue and fluorophore-to-antibody ratio (“FAR”) for various antibody conjugates.

FIG. 9 provides results of the conjugation, including amino acid residue and fluorophore-to-antibody ratio (“FAR”) for various antibody conjugates.

FIG. 10 provides results of the conjugation, including amino acid residue and fluorophore-to-antibody ratio (“FAR”) for various antibody conjugates.

FIG. 11 illustrates stability for various antibody conjugates.

FIG. 12A-C provide graphs of FACS Binding data for C6-CYSMAB-ADCs. Circle, C6-S156C-D202; square, C6-A118C-D202; triangle, C6-G316C-D202; open circle, C6-V266C-D202; open square, C6-S239C-D202; open triangle, C0-D202.

### DETAILED DESCRIPTION OF THE INVENTION

This application is not limited to particular methodologies or the specific compositions described, as such may vary. It is also to be understood that the terminology used herein is for the purpose of describing particular embodiments only, and is not intended to be limiting, since the scope of the present application will be limited only by the appended claims and their equivalents.

Unless defined otherwise, all technical and scientific terms used herein have the same meaning as commonly understood by one of ordinary skill in the art to which this disclosure belongs. Suggested methods and materials are described, hereafter, although any methods and materials similar or equivalent to those described herein can be used in the practice or testing of the present application.

### I. DEFINITIONS

An “immunoglobulin”, as used herein, refers to an immunoglobulin polypeptide or an antibody.

The term “immunoglobulin polypeptide” refers to a polypeptide substantially encoded by an immunoglobulin gene.

The term “antibody” refers to a protein having antigen binding activity and an amino acid sequence from or derived from the framework region of an immunoglobulin encoding gene of an animal producing antibodies. The term includes but is not limited to polyclonal or monoclonal antibodies of the isotype classes IgA, IgD, IgE, IgG, and IgM, derived from human or other mammalian cells, including natural or genetically modified forms such as humanized, human, single-chain, chimeric, synthetic, recombinant, hybrid, mutated, grafted, and in vitro generated antibodies. The term encompasses conjugates, including but not limited to fusion proteins containing an immunoglobulin moiety (e.g., chimeric or bispecific antibodies or scFv′s), and fragments, such as Fab, F(ab′)2, Fv, scFv, Fd, single domain (dAb) and other compositions.

The term “cysteine substituted immunoglobulin,” as used herein, refers to a cysteine substituted immunoglobulin polypeptide or cysteine substituted antibody.

The term “cysteine substituted immunoglobulin polypeptide” as used herein refers to a polypeptide comprising at least one non-naturally occurring constant region immunoglobulin amino acid residue that has been substituted with cysteine. A non-naturally occurring substitution is one that is not isotypic. In one embodiment, the substituted residues are heavy chain constant regions residues V266C, H285C, R301C, V303C, T307C, G316C, Y436C and L441C. In another embodiment the constant region is of isotype IgG1, IgG2, IgG3 or IgG4.

The term “cysteine substituted antibody” (“CYSMAB”) refers to an antibody comprising a cysteine substituted immunoglobulin polypeptide.

The terms “immunoglobulin conjugate” as used herein, refer to an immunoglobulin polypeptide or an antibody or “antibody conjugate” that is conjugated to a functional moiety.

The terms “immunoglobulin drug conjugate” as used herein, refers to an immunoglobulin polypeptide or antibody (“antibody drug conjugate” (“ADC”)), that is conjugated to a functional moiety, such as a drug moiety or a radiolabel or a detection reagent.

The term “Cysteine substituted immunoglobulin drug conjugate” or refers to a cysteine substituted immunoglobulin polypeptide or cysteine substituted antibody (“CYSMAB”) that has been conjugated to a drug moiety, e.g., cysteine substituted antibody drug conjugate (“CYSMAB ADC”).

An exemplary antibody immunoglobulin structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The variable region contains the antigen-binding region of the antibody (or its functional equivalent) and is most critical in specificity and affinity of binding. See Paul, Fundamental Immunology (2003).

Antibodies can exist as intact immunoglobulins or as any of a number of well-characterized fragments that include specific antigen-binding activity. For the sake of clarity, a tetrameric antibody with heavy and light chains is referred to herein as an “intact immunoglobulin,” and can be naturally occurring, polyclonal, monoclonal, or recombinantly produced. Fragments can be produced by digestion with various peptidases. Pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies or those identified using phage display libraries (see, e.g., McCafferty et al., Nature 348:552-554 (1990)).

As used herein, the term “Fv” refers to a monovalent or bi-valent variable region fragment, and can encompass only the variable regions (e.g., VL and/or VH), as well as longer fragments, e.g., an Fab, Fab′ or F(ab′)2, which also includes CL and/or CH1. Unless otherwise specified, the term “Fc” refers to a heavy chain monomer or dimer comprising CH2 and CH3 regions.

A single chain Fv (scFv) refers to a polypeptide comprising a VL and VH joined by a linker, e.g., a peptide linker. ScFvs can also be used to form tandem (or di-valent) scFvs or diabodies. Production and properties of tandem scFvs and diabodies are described, e.g., in Asano et al. (2011) J Biol. Chem. 286:1812; Kenanova et al. (2010) Prot Eng Design Sel 23:789; Asano et al. (2008) Prot Eng Design Sel 21:597.

The term “monoclonal antibody” as used herein refers to a clonal preparation of antibodies with a single binding specificity and affinity for a given epitope on an antigen. A “polyclonal antibody” refers to a preparation of antibodies that are raised against a single antigen, but with different binding specificities and affinities.

As used herein, “variable region” or “V-region” refers to an antibody variable region domain comprising the segments of Framework 1, CDR1, Framework 2, CDR2, and Framework 3, including CDR3 and Framework 4, which segments are added to the V-segment as a consequence of rearrangement of the heavy chain and light chain V-region genes during B-cell differentiation.

The term “framework” or “FR” as used herein refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-HVR1(L1)-FR2-HVR2(L2)-FR3-HVR3(L3)-FR4.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

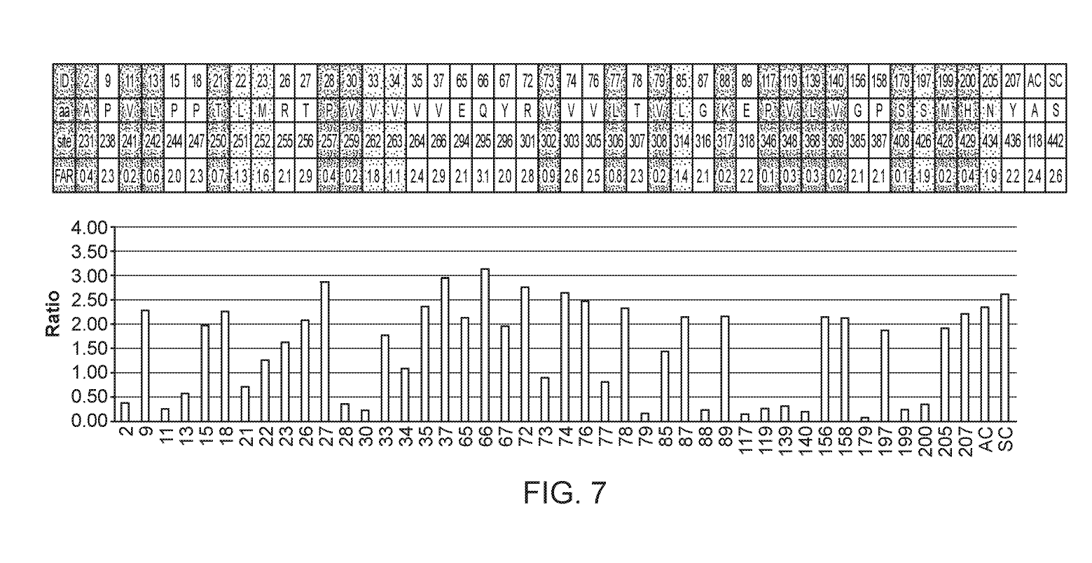

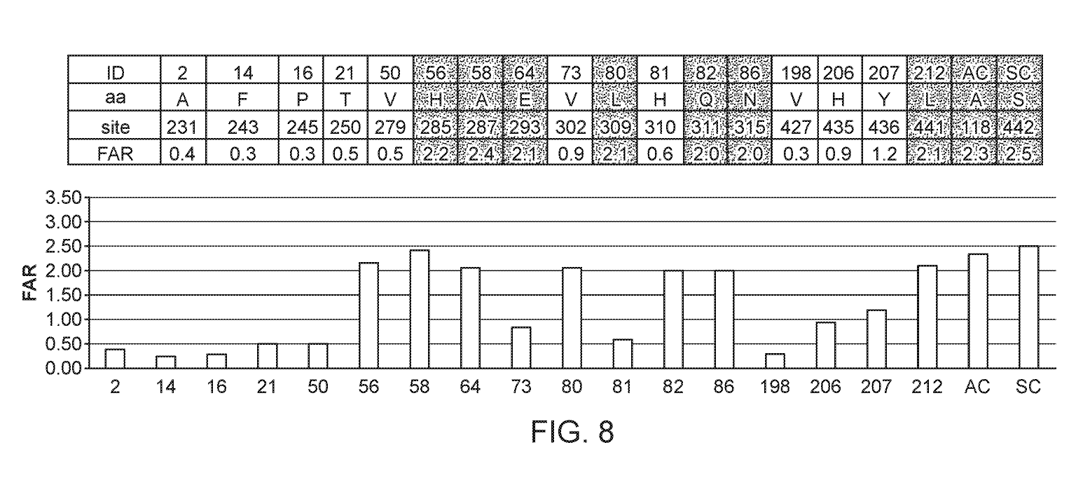

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, (1987) J. Mol. Biol. 196, 901-917; Chothia et al. (1989) Nature 342, 877-883; Chothia et al. (1992) J. Mol. Biol. 227, 799-817; Al-Lazikani et al., J. Mol. Biol. 1997, 273(4)). A helpful guide for locating CDRs using the Kabat system can be found at the website available at bioinf.org.uk/abs. Definitions of antigen combining sites are also described in the following: Ruiz et al. Nucleic Acids Res., 28, 219-221 (2000); and Lefranc Nucleic Acids Res. January 1; 29(1):207-9 (2001); MacCallum et al., J. Mol. Biol., 262: 732-745 (1996); and Martin et al, Proc. Natl. Acad. Sci. USA, 86, 9268-9272 (1989); Martin, et al, Methods Enzymol., 203: 121-153, (1991); Pedersen et al, Immunomethods, 1, 126, (1992); and Rees et al, In Sternberg M. J. E. (ed.), Protein Structure Prediction. Oxford University Press, Oxford, 141-172 1996). Example CDRs are described as CDR-H1, CDR-H2, CDR-H3, CDR-L1, CDR-L2 and CDR-L3 of FIG. 7 of US2013/0295118.

The term “hypervariable region”, “HVR” when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)), whereas Chothia refers to the location of the structural loops (Chothia and Lesk (1987) J. Mol. Biol. 196:901-917). The “contact” hypervariable regions are based on an analysis of the available complex crystal structures. The residues from each of these hypervariable regions are noted below. Unless otherwise denoted, Kabat numbering according to the Kabat Database of aligned sequences of proteins will be employed (Wu and Kabat (1970) J. Exp. Med. 132:211-250; Johnson and Wu (2000) Nuc. Acids Res. 28(1):214-218). Hypervariable region locations are generally as follows: amino acids 24-34 (HVR-L1), amino acids 49-56 (HVR-L2), amino acids 89-97 (HVR-L3), amino acids 26-35A (HVR-H1), amino acids 49-65 (HVR-H2), and amino acids 93-102 (HVR-H3). Hypervariable regions may also comprise “extended hypervariable regions” as follows: amino acids 24-36 (L1), and amino acids 46-56 (L2) in the VL. The variable domain residues are numbered according to Kabat et al., supra for each of these definitions. An “altered hypervariable region” for the purposes herein is a hypervariable region comprising one or more (e.g. one to about 16) amino acid substitution(s) therein. An “unmodified hypervariable region” for the purposes herein is a hypervariable region having the same amino acid sequence as a non-human antibody from which it was derived, i.e. one which lacks one or more amino acid substitutions therein.

The term “chimeric antibody” as used herein refers to an antibody in which (a) the constant region, or a portion thereof, is altered, replaced or exchanged so that the antigen binding site (variable region, CDR, or portion thereof) is linked to a constant region of a different or altered class, effector function and/or species; or (b) the variable region, or a portion thereof, is altered, replaced or exchanged with a variable region having a different or altered antigen specificity (e.g., CDR and framework regions from different species). Chimeric antibodies can include variable region fragments, e.g., a recombinant antibody comprising two Fab or Fv regions or an scFv. A chimeric can also, as indicated above, include an Fc region from a different source than the attached Fv regions. In some cases, the chimeric antibody includes chimerism within the Fv region. An example of such a chimeric antibody would be a humanized antibody where the FRs and CDRs are from different sources.

The term “Humanized antibodies” as used herein refers to antibodies in which the antigen binding loops, i.e., CDRs, obtained from the V_{H} and V_{L} regions of a non-human antibody are grafted to a human framework sequence. Humanization, i.e., substitution of non-human CDR sequences for the corresponding sequences of a human antibody, can be performed following the methods described in, e.g., U.S. Pat. Nos. 5,545,806; 5,569,825; 5,633,425; 5,661,016; Riechmann et al., Nature 332:323-327 (1988); Marks et al., Bio/Technology 10:779-783 (1992); Morrison, Nature 368:812-13 (1994); Fishwild et al., Nature Biotechnology 14:845-51 (1996). Transgenic mice, or other organisms such as other mammals, may also be used to express humanized or human antibodies, as disclosed in U.S. Pat. No. 6,673,986.

The terms “specific for,” “specifically binds,” and like terms refer to a molecule (e.g., antibody or antibody fragment) that binds to a target with at least 2-fold greater affinity than non-target compounds, e.g., at least any of 4-fold, 5-fold, 6-fold, 7-fold, 8-fold, 9-fold, 10-fold, 20-fold, 25-fold, 50-fold, or 100-fold greater affinity. For example, an antibody that specifically binds a primary target will typically bind the primary target with at least a 2-fold greater affinity than a non-primary antibody target (e.g., an antibody from a different species or of a different isotype, or a non-antibody target).

The term “binds” with respect to an antibody target (e.g., antigen, analyte, immune complex) typically indicates that an antibody binds a majority of the antibody targets in a pure population (assuming appropriate molar ratios). For example, an antibody that binds a given antibody target typically binds to at least ⅔ of the antibody targets in a solution (e.g., at least any of 75, 80, 85, 90, 91, 92, 93, 94, 95, 96, 97, 98, 99, or 100%). One of skill will recognize that some variability will arise depending on the method and/or threshold of determining binding.

The terms “label,” “detectable moiety,” and like terms refer to a composition detectable by spectroscopic, photochemical, biochemical, immunochemical, chemical, or other physical means. For example, useful labels include fluorescent dyes. luminescent agents, radioisotopes (e.g., ³²P, ³H), electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target analyte. Any method known in the art for conjugating an antibody to the label may be employed, e.g. using methods described in Hermanson, Bioconjugate Techniques 1996, Academic Press, Inc., San Diego. The term “tag” can be used synonymously with the term “label,” but generally refers to an affinity-based moiety, e.g., a “His tag” for purification, or a “streptavidin tag” that interacts with biotin.

The term “labeled” molecule (e.g., nucleic acid, protein, or antibody) as used herein is one that is bound, either covalently, through a linker or a chemical bond, or non-covalently, through ionic, van der Waals, electrostatic, or hydrogen bonds to a label such that the presence of the molecule may be detected by detecting the presence of the label bound to the molecule.

The term “C-type lectin-Like molecule 1 (CLL-1),” also known as CLEC12A, DCAL-2, and MICL, is a type II membrane protein (ITIM domain—TM domain-stalk domain-lectin-like domain). The extracellular domain of CLL-1 is highly glycosylated, and it is expressed exclusively in cells of myeloid lineage. CLL-1 is also expressed on AML, MDS, and CML cells. CLL-1 expression can be used to distinguish between normal hematopoietic stem cells (HSCs), which do not express CLL-1, and leukemic stein cells (LSCs), where it is expressed. LSCs are CD34+ cells in leukemia patients that lead to production of cancer cells and recurrence of cancer. See Bakker et al. (2004) Cancer Res. 64:8443.

The nucleotide and amino acid sequences of CLL-1 are known for many species. For example, the human sequences can be found as SEQ ID NO:2 in US2013/0295118 and Genbank accession number AF247788.1 and Uniprot accession number Q5QGZ9 (SEQ ID NO:2). For the human CLL-1 protein shown as SEQ ID NO:2, the extracellular domain comprises approximately amino acids 65-265, the transmembrane domain comprises approximately amino acids 44-64, and the cytoplasmic domain comprises approximately amino acids 1-43. The stalk domain of human CLL-1 spans amino acids 65-139, and the C lectin domain spans amino acids 140-249

The term “CLL-1 associated disorder” as used herein refers to conditions and diseases correlated with non-pathogenic levels (e.g., elevated or reduced cell surface expression of CLL-1) as compared to CLL-1 expression in a standard control (e.g., a normal, non-disease, non-cancer cell). Elevated CLL-1 levels are associated with cancer cells, in particular, leukemias such as AML (acute myelogenous leukemia), MDS (myelodysplastic syndrome), and CML (chronic myelogenous leukemia), and in hematopoietic CSCs (e.g., LSCs).

The “cancer stem cell” hypothesis proposes that only a small portion of a tumor is represented by the “cancer stem cell,” which allows the tumor to proliferate and self-renew, and eventually differentiate into the phenotypically diverse and heterogeneous tumor cell population (Bjerkvig et al., Nat. Rev. Cancer, 5:899-904, 2005). Cancer stem cells can be isolated from any type of cancers, e.g., leukemias, breast, colon and brain cancers, colon cancers. Cancer stern cells are characterized by their ability to self-renew and proliferate, and recapitulate through differentiation from the parental tumor. Exemplary cancer stem cell antigens include CD133, Bmi-1, Notch, Sonic hedgehog, and Wnt. Additionally, exemplary molecular markers of neural cancer stem cells include CD90, CD44, CXCR4, Nestin, Musashi-1 (Msi1), maternal embryonic leucine zipper kinase (MELK), GLI1, PTCH1, Bmi-1, phosphoserine phosphatase (PSP). Snail, OCT4, BCRP1, MGMT, Bcl-2, FLIP, BCL-XL, XIAP, cIAP1, cIAP2, NAIP, and survivin. One useful cancer stem cell antigen is CLL-1.

The term “cytotoxic” refers to the inhibitory effect that an agent has on a cell, e.g., necrosis (a loss of membrane integrity and rapid death as a result of cell lysis); decreased viability (wherein cells stop proliferating) and apoptosis (a genetic program of controlled cell death).

Cytotoxicity can also be monitored using the 3-(4,5-Dimethyl-2-thiazolyl)-2,5-diphenyl-2H-tetrazolium bromide (MTT) or MTS assay. This assay measures the reducing potential of the cell using a colorimetric reaction. Viable cells will reduce the MTS reagent to a colored formazan product. A similar redox-based assay has also been developed using the fluorescent dye, resazurin. In addition to using dyes to indicate the redox potential of cells in order to monitor their viability, researchers have developed assays that use adenosine triphosphate (ATP) content as a marker of viability. Such ATP-based assays include bioluminescent assays in which ATP is the limiting reagent for the luciferase reaction (e.g. CellTiter-Glo Luminescent Cell Viability assay, Promega). Cytotoxicity can also be measured by the sulforhodamine B (SRB) assay, WST assay and chronogenic assay. A label-free approach to follow the cytotoxic response of adherent animal cells in real-time is based on electric impedance measurements when the cells are grown on gold-film electrodes. This technology is referred to as electric cell-substrate impedance sensing (ECIS). Label-free real-time techniques provide the kinetics of the cytotoxic response rather than just a snapshot like many colorimetric endpoint assays.

The terms “CLL-1 specific antibody,” “anti-CLL-1 antibody,” “CLL-1 antibody,” and “anti.-CLL-1” are used synonymously herein to refer to an antibody that specifically binds to CLL-1, including variously glycosylated forms of CLL-1. The CLL-1 antibodies described herein specifically bind the CLL-1 polypeptide expressed, e.g., on the surface of certain cancer cells, but not to hematopoietic stem cells (HSCs). As discussed in more detail below, the present anti-CLL-1 antibodies can bind CLL-1 expressing cells, bind a larger percentage of AML cells compared to other AML-targeting antibodies, inhibit AML cell proliferation, and mediate their destruction. Examples of anti-CLL antibodies suitable for use as cysteine substituted antibodies (CYSMABs) of this disclosure are described US2013/0295118, published Nov. 7, 2013. An anti-CLL-1 antibody can have CDRs as disclosed in that publication, in particular, CDRs for antibodies M31 and M26.

The term “differentially expressed” or “differentially regulated” refers generally to a protein or nucleic acid biomarker that is overexpressed (upregulated) or under-expressed (downregulated) in one sample compared to at least one other sample. In the context of the present disclosure, the term generally refers to overexpression of CLL-1 on a cancer cell (e.g., an AML cell or AML CSC) compared to a normal, non-cancer cell.

For example, the terms “overexpressed” or “unregulated” interchangeably refer to a protein or nucleic acid, generally a biomarker, that is transcribed or translated at a detectably greater than control level. The term includes overexpression due to transcription, post transcriptional processing, translation, post-translational processing, cellular localization (e.g., organelle, cytoplasm, nucleus, cell surface), and RNA and protein stability. Overexpression can be detected using conventional techniques for detecting biomarkers, whether mRNA (i.e., RT-PCR, hybridization) or protein (i.e., flow cytometry, imaging, ELISA, immunohistochemical techniques). Overexpression can be at least any of 10%, 20%, 30%, 40%, 50%, 60%, 70%, 80%, 90% or more in comparison to a normal cell.

The term “control” sample or value as used herein refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). In the context of the present disclosure, an example of a negative control would be a biological sample from a known healthy (non-cancer) individual, and an example of a positive control would be a biological sample from a known AML patient. A control can also represent an average value or a range gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “diagnosis” refers to a relative probability that a subject has a disorder such as cancer. Similarly, the term “prognosis” refers to a relative probability that a certain future outcome may occur in the subject. For example, in the context of the present disclosure, prognosis can refer to the likelihood that an individual will develop cancer, have recurrence, or the likely severity of the disease (e.g., severity of symptoms, rate of functional decline, survival, etc.). The terms are not intended to be absolute, as will be appreciated by any one of skill in the field of medical diagnostics.

“Biopsy” or “biological sample from a subject” as used herein refers to a sample obtained from a subject having, or suspected of having a disease, e.g., a CLL-1 associated disorder. The sample can also be a blood sample or blood fraction, white blood cell fraction, serum, or plasma. In some embodiments, the sample may be a tissue biopsy, such as needle biopsy, fine needle biopsy, surgical biopsy, etc. The sample can comprise a tissue sample harboring a lesion or suspected lesion, although the biological sample may be also be derived from another site, e.g., a site of suspected metastasis, a lymph node, or from the blood. In some cases, the biological sample may also be from a region adjacent to the lesion or suspected lesion.

A “biological sample” can be obtained from a subject, e.g., a biopsy, from an animal, such as an animal model, or from cultured cells, e.g., a cell line or cells removed from a subject and grown in culture for observation. Biological samples include tissues and bodily fluids, e.g., blood, blood fractions, lymph, saliva, urine, feces, etc.

The EU numbering scheme refers to the number of the US antibody (Edelman et al., Proc. Natl. Acad. Sci. USA 63: 78-85 (1969)). The Kabat Complementarity Determining Regions (CDRs) are based on sequence variability and are the most commonly used (Kabat et al. Sequences of Proteins of Immunological Interest, 5th Ed. Public Health Service, National institutes of Health, Bethesda, Md. (1991)). As used herein, EU number refers to the constant chain nomenclature of the antibodies described herein, while Kabat is used to derive the CDRs and HVRs of the variable regions.

“Operably linked” refers to a juxtaposition of two or more components, wherein the components so described are in a relationship permitting them to function in their intended manner. For example, a promoter is operably linked to a coding sequence if it acts in cis to control or modulate the transcription of the linked sequence. Generally, but not necessarily, the DNA sequences that are “operably linked” are contiguous and, where necessary to join two protein coding regions or in the case of a secretory leader, contiguous and in reading frame. However, although an operably linked promoter is generally located upstream of the coding sequence, it is not necessarily contiguous with it.

The term “promoter” as used herein refers to a polynucleotide sequence that controls transcription of a gene or sequence to which it is operably linked. A promoter includes signals for RNA polymerase binding and transcription initiation. The promoters used will be functional in the cell type of the host cell in which expression of the selected sequence is contemplated.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

The term “host cell” (or “recombinant host cell”), as used herein, is intended to refer to a cell that has been genetically altered, or is capable of being genetically altered by introduction of an exogenous polynucleotide, such as with a recombinant plasmid or vector. It should be understood that such terms refer not only to the particular subject cell but also to its progeny.

The terms “therapy,” “treatment,” and “amelioration” refer to any reduction in the severity of symptoms. In the case of treating cancer (e.g., AML), treatment can refer to, e.g., reducing tumor size, number of cancer cells, growth rate, metastatic activity, reducing cell death of non-cancer cells, reduced nausea and other chemotherapy or radiotherapy side effects, etc. The terms “treat” and “prevent” are not intended to be absolute terms. Treatment and prevention can refer to any delay in onset, amelioration of symptoms, improvement in patient survival, increase in survival time or rate, etc. Treatment and prevention can be complete (undetectable levels of neoplastic cells) or partial, such that fewer neoplastic cells are found in a patient than would have occurred without the present invention. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

An “effective amount” of an agent, e.g., a pharmaceutical formulation, refers to an amount effective, at dosages and for periods of time necessary, to achieve the desired cellular response, therapeutic or prophylactic result. For example, in a method for inhibiting cell proliferation, an effective amount of a cysteine substituted immunoglobulin drug conjugate (e.g., CYSMAB ADC) is a concentration which noticeably attenuates, inhibits, or prevents cell division in a cell relative to a control cell.

The phrase “therapeutically effective amount” means an amount of a compound of the present invention that (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. In one embodiment, the therapeutic effective amount is an amount sufficient to decrease or alleviate the symptoms of a disorder responsive to the modulation of CLL-1. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR). In one embodiment, the therapeutic effective amount is an amount sufficient to decrease or alleviate the symptoms of a disorder responsive to the modulation of CLL-1. In the case of immunological disorders, the therapeutic effective amount is an amount sufficient to decrease or alleviate an allergic disorder, the symptoms of an autoimmune and/or inflammatory disease, or the symptoms of an acute inflammatory reaction. In some embodiments, a therapeutically effective amount is an amount of a chemical entity described herein sufficient to significantly decrease the activity or number of myeloproliferative cancer stem cells.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

The phrase “pharmaceutically acceptable salt,” as used herein, refers to pharmaceutically acceptable organic or inorganic salts of an ADC. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

“Pharmaceutically acceptable solvate” refers to an association of one or more solvent molecules and an ADC. Examples of solvents that form pharmaceutically acceptable solvates include, but are not limited to, water, isopropanol, ethanol, methanol, DMSO, ethyl acetate, acetic acid, and ethanolamine.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®. The terms “dose” and “dosage” are used interchangeably herein. A dose refers to the amount of active ingredient given to an individual at each administration. For the present invention, the dose can refer to the concentration of the antibody or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease. The term “patient” refers to a subject under medical supervision. A patient can be an individual that is seeking treatment, monitoring, adjustment or modification of an existing therapeutic regimen, etc. A “cancer patient” or “AML patient” can refer to an individual that has been diagnosed with cancer, is currently following a therapeutic regimen, or is at risk of recurrence, e.g., after surgery to remove a tumor. In some embodiments, the cancer patient has been diagnosed with cancer and is a candidate for therapy. Cancer patients can include individuals that have not received treatment, are currently receiving treatment, have had surgery, and those that have discontinued treatment.

In the context of treating cancer, a subject in need of treatment can refer to an individual that has cancer or a pre-cancerous condition, has had cancer and is at risk of recurrence, is suspected of having cancer, is undergoing standard treatment for cancer, such as radiotherapy or chemotherapy, etc.

“Cancer”, “tumor,” and like terms include precancerous, neoplastic, and cancerous cells, and can refer to a solid tumor, or a non-solid cancer (see, e.g., Edge et al. AJCC Cancer Staging Manual (7^{th} ed. 2009); Cibas and Ducatman Cytology: Diagnostic principles and clinical correlates (3^{rd} ed. 2009)). Cancer includes both benign and malignant neoplasms (abnormal growth).

The term “cancer” can refer to leukemias, carcinomas, sarcomas, adenocarcinomas, lymphomas, solid and lymphoid cancers, etc. Examples of different types of cancer include, but are not limited to, acute my elogenous leukemia (AML), chronic myelogenous leukemia, (CML), B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, multiple myelomas, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, pancreatic cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma; head and neck cancer, osteogenic sarcoma, fibrosarcoma, neuroblastoma, glioma, and melanoma.

A “cancer target” or “cancer marker” is a molecule that is differentially expressed or processed in cancer, e.g., on a cancer cell or in the cancer milieu. Exemplary cancer targets are cell surface proteins such as CLL-1 (also, e.g., cell adhesion molecules and receptors), intracellular receptors, hormones, and molecules such as proteases that are secreted by cells into the cancer milieu. Markers for specific cancers are known in the art. e.g., CD45 for AML, CD34±CD38—for AML CSCs, MUC1 expression on colon and colorectal cancers, bombesin receptors in lung cancer, and prostate specific membrane antigen (PSMA) on prostate cancer.

In some embodiments, the cancer target can be associated with a certain type of cancer cell, e.g., AML, leukemia, myeloma, lymphoma, non-small cell lung cancer cells, prostate cancer, colorectal cancer, breast cancer or ovarian cancer. A cell type specific target is typically expressed at levels at least 2 fold greater in that cell type than in a reference population of cells. In some embodiments, the cell type specific marker is present at levels at least any of 3, 4, 5, 6, 7, 8, 9, 10, 20, 50, 100, or 1000 fold higher than its average expression in a reference population. Thus, the target can be detected or measured to distinguish the cell type or types of interest from other cells. For example, AML cancer targets include CLL-1, Ly86, LILRA1, and CD180.

A cancer stem cell (CSC) is a cell found in a tumor or blood cancer that can give rise to the cells that make up the bulk of the cancer. The CSC can also be self-renewing, similar to a normal (non-cancer) stem cell. CSCs can thus mediate metastasis by migrating to a non-tumor tissue in an individual and starting a “new” tumor. CSCs make up a very small percentage of any given cancer, depending on the stage that the cancer is detected. For example, the average frequency of CSCs in a sample of AML cells is believed to be about 1:10,000. Hematopoietic CSCs can be identified as CD34+, similar to normal hematopoietic stem cells (HSCs).

“Conservatively modified variants” applies to both amino acid and nucleic acid sequences. With respect to particular nucleic acid sequences, conservatively modified variants refers to those nucleic acids which encode identical amino acid sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide are implicit in a described sequence with respect to the expression product, but not with respect to actual probe sequences.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous,” with reference to a polynucleotide or polypeptide, indicates that the polynucleotide or polypeptide comprises two or more subsequences that are not found in the same relationship to each other in nature. For instance, a heterologous polynucleotide or polypeptide is typically recombinantly produced, having two or more sequences from unrelated genes arranged to make a new functional unit, e.g., a promoter from one source and a coding region from another source. Similarly, a heterologous protein indicates that the protein comprises two or more subsequences that are not found in the same relationship to each other in nature (e.g., a fusion protein).

A “thiol reactive reagent” is a reagent having a moiety that reacts with a thiol to form a covalent bond. Thiol reactive reagents can have a group selected from halide, maleimide and sulfonyl. Non-limiting examples include biotin-PEO-maleimide ((+)-biotinyl-3-maleimidopropionamidyl-3,6-dioxaoctainediamine, Oda et al (2001) Nature Biotechnology 19:379-382, Pierce Biotechnology, Inc.) Biotin-BMCC, PEO-Iodoacetyl Biotin, Iodoacetyl-LC-Biotin, and Biotin-HPDP (Pierce Biotechnology, Inc.), and Nα-(3-maleimidylpropionyl)biocytin (MPB, Molecular Probes, Eugene, Oreg.). Other commercial sources for biotinylation, bifunctional and multifunctional linker reagents include Molecular Probes, Eugene, Oreg., and Sigma, St. Louis, Mo.

### II. POLYNUCLEOTIDES ENCODING CYSTEINE SUBSTITUTED IMMUNOGLOBULINS (E.G., CYSMAB)

Also provided are polynucleotides (e.g., DNA) encoding the cysteine substituted immunoglobulins described herein, or constant domains thereof having the cysteine substitution. Polynucleotides encoding cysteine substituted immunoglobulins can be prepared by site-directed mutagenesis on polynucleotides encoding immunoglobulin polypeptides. Kits for performing site directed mutagenesis are commercially available from a variety of sources. These include, for example, Phusion available from Life Technologies, QuikChange, available from Agilent Technologies, and Q5, available from New England Biolabs. In general, site directed mutagenesis involves primer extension of a target immunoglobulin-encoding polynucleotide using a primer that includes a mutant inserting a cys codon at the desired site.

Also provided are expression cassettes comprising a promoter operably linked to a polynucleotide encoding the cysteine substituted immunoglobulins described herein, or a constant domains thereof having the cysteine substitution. In some embodiments, the promoter is heterologous, i.e., not found in nature operably-linked to the coding sequence. In some embodiments, vectors (including but not limited to expression vectors or shuttle vectors) comprising a polynucleotide encoding the cysteine substituted immunoglobulins described herein, or a constant domains thereof having the cysteine substitution. Also provided are cells comprising, and optionally expressing, a polynucleotide encoding the cysteine substituted immunoglobulins described herein, or a constant domains thereof having the cysteine substitution. Exemplary cells include prokaryotic cells, including but not limited to E. coli, and eukaryotic cells, including but not limited to mammalian (e.g., human, hamster, rat, mouse, etc.), fungal (e.g., yeast), or plant cells.

### III. METHOD OF MAKING ANTIBODIES

For preparation of the presently described immunoglobulins, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this disclosure. Also, transgenic mice, or other organisms such as other mammals, can be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996), and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or under the control of different promoters. In other embodiments, the V_{H} and V_{L} region may be expressed using separate vectors. A V_{H} or V_{L} region as described herein may optionally comprise a methionine at the N-terminus.

An antibody of the disclosure can also be produced in various formats, including as a Fab, a Fab′, a F(ab′)₂, a scFv, or a dAb. The antibody fragments can be obtained by a variety of methods, including, digestion of an intact antibody with an enzyme, such as pepsin (to generate (Fab′)₂ fragments) or papain (to generate Fab fragments); or de novo synthesis. Antibody fragments can also be synthesized using recombinant DNA methodology. In some embodiments, the CLL-1 antibody comprises F(ab′)₂ fragments that specifically bind CLL-1. An antibody of the disclosure can also include a human constant region. See, e.g., Fundamental Immunology (Paul ed., 4d ed. 1999); Bird, et al., Science 242:423 (1988); and Huston, et al., Proc. Natl. Acad. Sci. USA 85:5879 (1988).

Methods for humanizing non-human antibodies (i.e., using CDRs from non-human antibodies) are also known in the art. Generally, a humanized antibody has one or more amino acid residues from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol., 2:593-596 (1992)), by substituting rodent CDRs or CDR sequences for the corresponding sequences of a human antibody. Such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

In some cases, the antibody or antibody fragment can be conjugated to another molecule, e.g., polyethylene glycol (PEGylation) or serum albumin, to provide an extended half-life in vivo. Examples of PEGylation of antibody fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20: 227 2007). The antibody or antibody fragment can also be labeled, or conjugated to a therapeutic agent as described below.

### IV. PREPARATION OF CYSTEINE SUBSTITUTED IMMUNOGLOBULIN (E.G., CYSMAB) DRUG CONJUGATES

Antibody-Drug Conjugates prepared from the cysteine substituted immunoglobulins (CYSMABs) of the disclosure may be prepared by several routes, employing organic chemistry reactions, conditions, and reagents known to those skilled in the art, including: (1) reaction of a cysteine group of a cysteine engineered antibody with a linker reagent, to form antibody-linker intermediate Ab-L, via a covalent bond, followed by reaction with an activated drug moiety D; and (2) reaction of a nucleophilic group of a drug moiety with a linker reagent, to form drug-linker intermediate D-L, via a covalent bond, followed by reaction with a cysteine group of a cysteine engineered antibody (CYSMAB). Conjugation methods (1) and (2) may be employed with a variety of cysteine engineered antibodies (CYSMABs), drug moieties, and linkers to prepare the antibody-drug conjugates (ADCs).

Antibody cysteine thiol groups are nucleophilic and capable of reacting to form covalent bonds with electrophilic groups on linker reagents and drug-linker intermediates including: (i) active esters such as NHS esters, HOBt esters, haloformates, and acid halides; (ii) alkyl and benzyl halides, such as haloacetamides; (iii) aldehydes, ketones, carboxyl, and maleimide groups; and (iv) disulfides, including pyridyl disulfides, via sulfide exchange. Nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents.

Cysteine engineered antibodies may be made reactive for conjugation with linker reagents by treatment with a reducing agent such as DTT (Cleland's reagent, dithiothreitol) or TCEP (tris(2-carboxyethyl)phosphine hydrochloride; Getz et al (1999) Anal. Biochem. Vol. 273:73-80; Soltec Ventures, Beverly, Mass.), followed by reoxidation, e.g., with DHAA to reform inter-chain and intra-chain disulfide bonds (Example 2).

A. Linkers

“Linker”, “Linker Unit”, or “link” means a chemical moiety comprising a covalent bond or a chain of atoms that covalently attaches an antibody to a drug moiety. In various embodiments, a linker is specified as: L. A “Linker” (L) is a bifunctional or multifunctional moiety which can be used to link one or more Drug moieties (D) and an antibody unit (Ab) to form antibody-drug conjugates (ADCs). Antibody-drug conjugates (ADCs) can be conveniently prepared using a Linker having reactive functionality for binding to the Drug and to the Antibody. A cysteine thiol of a cysteine engineered antibody (CYSMAB) can form a bond with an electrophilic functional group of a linker reagent, a drug moiety or drug-linker intermediate.

In one aspect, a Linker has a reactive site, which has an electrophilic group that is reactive to a nucleophilic cysteine present on an antibody. The cysteine thiol of the antibody is reactive with an electrophilic group on a Linker and forms a covalent bond to a Linker. Useful electrophilic groups include, but are not limited to, maleimide and haloacetamide groups.

Linkers include a divalent radical such as an alkyldiyl, an arylene, a heteroarylene, moieties such as: —(CR₂)ₙO(CR₂)ₙ—, repeating units of alkyloxy (e.g. polyethylenoxy, PEG, polymethyleneoxy) and alkylamino (e.g. polyethyleneamino, Jeffamine™); and diacid ester and amides including succinate, succinamide, diglycolate, malonate, and caproamide.

Cysteine engineered antibodies (CYSMABs) react with linker reagents or drug-linker intermediates, with electrophilic functional groups such as maleimide or α-halo carbonyl, according to the conjugation method at page 766 of Klussman, et al (2004), Bioconjugate Chemistry 15(4):765-773.

The linker may be composed of one or more linker components. Exemplary linker components include 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), N-succinimidyl 4-(2-pyridylthio) pentanoate (“SPP”), N-succinimidyl 4-(N-maleimidomethyl) cyclohexane-1 carboxylate (“SMCC”), N-Succinimidyl (4-iodo-acetyl) aminobenzoate (“SIAB”), ethyleneoxy —CH2CH2O— as one or more repeating units (“EO” or “PEO”). Additional linker components are known in the art and some are described herein.

In another embodiment, a Linker has a reactive functional group, which has a nucleophilic group that is reactive to an electrophilic group present on an antibody. Useful electrophilic groups on an antibody include, but are not limited to, aldehyde and ketone carbonyl groups. The heteroatom of a nucleophilic group of a Linker can react with an electrophilic group on an antibody and form a covalent bond to an antibody unit. Useful nucleophilic groups on a Linker include, but are not limited to, hydrazide, oxime, amino, hydrazine, thiosemicarbazone, hydrazine carboxylate, and arylhydrazide. The electrophilic group on an antibody provides a convenient site for attachment to a Linker.

Typically, peptide-type Linkers can be prepared by forming a peptide bond between two or more amino acids and/or peptide fragments. Such peptide bonds can be prepared, for example, according to the liquid phase synthesis method (E. Schröder and K. Lübke (1965) “The Peptides”, volume 1, pp 76-136, Academic Press), which is well known in the field of peptide chemistry. Linker intermediates may be assembled with any combination or sequence of reactions including spacer, stretcher, and amino acid units. The spacer, stretcher, and amino acid units may employ reactive functional groups which are electrophilic, nucleophilic, or free radical in nature. Reactive functional groups include, but are not limited to carboxyls, hydroxyls, para-nitrophenylcarbonate, isothiocyanate, and leaving groups, such as O-mesyl, O-tosyl, —Cl, —Br, —I; or maleimide.

In another embodiment, the Linker may be substituted with groups, which modulated solubility or reactivity. For example, a charged substituent such as sulfonate (—SO3-) or ammonium, may increase water solubility of the reagent and facilitate the coupling reaction of the linker reagent with the antibody or the drug moiety, or facilitate the coupling reaction of Ab-L (antibody-linker intermediate) with D, or D-L (drug-linker intermediate) with Ab, depending on the synthetic route employed to prepare the ADC.

An exemplary phe-lys(Mtr, mono-4-methoxytrityl)dipeptide linker reagent comprising a maleimide moiety and a PAB self-immolative moiety can be prepared according to Dubowchik, et al. (1997) Tetrahedron Letters, 38:5257-60.

B. Linker Reagents

Conjugates of the antibody and auristatin may be made using a variety of bifunctional linker reagents such as N-succinimidyl-3-(2-pyridyldithio)propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

Linker reagents useful for the antibody drug conjugates (ADCs) of the disclosure include, but are not limited to: BMPEO, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate), and including bis-maleimide reagents: DTME, BMB, BMDB, BMH, BMOE, 1,8-bis-maleimidodiethyleneglycol (BM(PEO)2), and 1,11-bis-maleimidotriethyleneglycol (BM(PEO)3), which are commercially available from Pierce Biotechnology, Inc., ThermoScientific, Rockford, Ill., and other reagent suppliers. Bis-maleimide reagents allow the attachment of a free thiol group of a cysteine residue of an antibody to a thiol-containing drug moiety, label, or linker intermediate, in a sequential or concurrent fashion. Other functional groups besides maleimide, which are reactive with a thiol group of an antibody, nemorubicin metabolite and analog drug moiety, or linker intermediate include iodoacetamide, bromoacetamide, vinyl pyridine, disulfide, pyridyl disulfide, isocyanate, and isothiocyanate.

V. Methods of Use

Cysteine substituted immunoglobulins of this disclosure are useful, among other things, in the preparation cysteine substituted immunoglobulin conjugates, including molecules conjugated to detectable moieties or drugs, such as cytotoxic agents.

A. Treatment of Disease

Cysteine substituted immunoglobulin drug conjugates (e.g., CYSMAB ADCs) are useful in the treatment of any disease treatable by targeting a cell to which such a conjugate binds. This includes any form of cancer.

Cysteine substituted immunoglobulin drug conjugates may be used to treat various diseases or disorders, e.g. characterized by the overexpression of a tumor antigen. Exemplary conditions or hyperproliferative disorders include benign or malignant tumors; leukemia and lymphoid malignancies. Others include neuronal, glial, astrocytal, hypothalamic, glandular, macrophagal, epithelial, stromal, blastocoelic, inflammatory, angiogenic and immunologic, including autoimmune, disorders.

Cysteine substituted immunoglobulin drug conjugates can be further tested in tumor-bearing higher primates and human clinical trials. Human clinical trials can be designed similar to the clinical trials testing the efficacy of the anti-HER2 monoclonal antibody HERCEPTIN® in patients with HER2 overexpressing metastatic breast cancers that had received extensive prior anti-cancer therapy as reported by Baselga et al. (1996) J. Clin. Oncol. 14:737-744. The clinical trial may be designed to evaluate the efficacy of an ADC in combinations with known therapeutic regimens, such as radiation and/or chemotherapy involving known chemotherapeutic and/or cytotoxic agents.

Generally, the disease or disorder to be treated is a hyperproliferative disease such as cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The cancer may comprise HER2-expressing cells, such that the ADC of the present invention are able to bind to the cancer cells. To determine ErbB2 expression in the cancer, various diagnostic/prognostic assays are available. In one embodiment, ErbB2 overexpression may be analyzed by IHC, e.g. using the HERCEPTEST (Dako). Paraffin embedded tissue sections from a tumor biopsy may be subjected to the IHC assay and accorded a ErbB2 protein staining intensity criteria as follows: Score 0, no staining is observed or membrane staining is observed in less than 10% of tumor cells; Score 1+, a faint/barely perceptible membrane staining is detected in more than 10% of the tumor cells, the cells are only stained in part of their membrane; Score 2+, a weak to moderate complete membrane staining is observed in more than 10% of the tumor cells; Score 3+, a moderate to strong complete membrane staining is observed in more than 10% of the tumor cells. Those tumors with 0 or 1+ scores for ErbB2 overexpression assessment may be characterized as not overexpressing ErbB2, whereas those tumors with 2+ or 3+ scores may be characterized as overexpressing ErbB2.

Alternatively, or additionally, FISH assays such as the INFORM™ (Ventana Co., Ariz.) or PATHVISION™ (Vysis, Ill.) may be carried out on formalin-fixed, paraffin-embedded tumor tissue to determine the extent (if any) of ErbB2 overexpression in the tumor.

Autoimmune diseases for which the ADC compounds may be used in treatment include rheumatologic disorders (such as, for example, rheumatoid arthritis, Sjögren's syndrome, scleroderma, lupus such as SLE and lupus nephritis, polymyositis/dermatomyositis, cryoglobulinemia, anti-phospholipid antibody syndrome, and psoriatic arthritis), osteoarthritis, autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-associated vasculitis, including Churg-Strauss vasculitis, Wegener's granulomatosis, and polyarteriitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)). More preferred such diseases include, for example, rheumatoid arthritis, ulcerative colitis, ANCA-associated vasculitis, lupus, multiple sclerosis, Sjögren's syndrome, IDDM, pernicious anemia, thyroiditis, and glomerulonephritis.

The presently described cysteine substituted immunoglobulin conjugates can also be used to detect and treat CLL-1 associated disorders, i.e., diseases correlated with elevated or reduced cell surface expression of CLL-1 as compared to CLL-1 expression in a standard control (e.g., a normal, non-disease, non-cancer cell). CLL-1 expression is normally limited to myeloid lineage cells, e.g., dendritic cells, granulocytes, and monocytes in the peripheral blood and spleen. Elevated CLL-1 levels are associated with cancer, in particular, in hematopoietic CSCs (e.g., LSCs), and in myeloproliferative disorders, including leukemias such as AML (acute myelogenous or myeloproliferative leukemia), MDS (myelodysplastic syndrome), myelofibrosis, CMML (chronic myelomonocytic leukemia), multiple myeloma, plasmacytoma, and CML (chronic myelogenous or myeloproliferative leukemia). See Bakker et al. (2004) Cancer Res. 64:8443; Van Rhenen et al. (2007) Blood 110:2659-66; Zhao et al. (2010) Haematologica (2010) 95:71; Van Rhenen et al. (2007) Leukemia 21:1700; and Herrmann et al. (2012) Haematologica 97:219.

AML cells can be characterized and distinguished from other cells by detecting cell surface marker expression. Aside from being CLL-1+, AML cells can be CD33+ (though some are CD33−), CD45+, and CDw52+. AML blasts (including LSCs) are typically CD34+CD38−. HSCs and LSCs can be characterized by expression of CD34, but the former do not express CLL-1. MDS cells can be characterized by expression of CD5, CD7, CD13, and CD34. CML cells can be characterized by expression of 7-ADD, CD33, CD34, and CD38.

Myelodysplastic Syndromes (MDS) include a group of closely-related blood formation disorders, in which the bone marrow shows qualitative and quantitative changes suggestive of a preleukemic process, but having a chronic course that does not necessarily terminate as acute leukemia. A variety of terms, including preleukemia, refractory anemia, refractory dysmyelopoietic anemia, smoldering or subacute leukemia, dysmyelopoietic syndrome (DMPS), and myelodysplasia, have all been used to describe MDS. These conditions are all characterized by a cellular marrow with impaired maturation (dysmyelopoiesis) and a reduction in the number of blood cells. DMPS is characterized by presence of megablastoids, megarkaryocyte dysplasia, and an increase in number of abnormal blast cells, reflective of enhanced granulocyte maturation process. Patients with DMPS show chromosomal abnormalities similar to those found in acute myeloid leukemia and progress to acute myeloid leukemia in a certain fraction of afflicted patients.

Chronic myeloproliferative disorders are a collection of conditions characterized by increased number of mature and immature granulocytes, erythrocytes, and platelets. Chronic myeloproliferative disorders can transition to other forms within this group, with a tendency to terminate in acute myeloid leukemia. Specific diseases within this group include polycythemia vera, chronic myeloid leukemia, agnogenic myeloid leukemia, essential thrombocythemia, and chronic neutrophilic leukemia.

Myelofibrosis is characterized by scarring of the bone marrow that results in reduced number of red and white blood cells, and platelets. Myelofibrotic scarring can result from leukemia, but can have other causes, such as thrombocytosis or adverse drug effects.

B. CDC, ADCC, and ADC Assays

The effectiveness of the cysteine substituted immunoglobulin drug conjugates (e.g., CYSMAB ADCs) of the disclosure can be evaluated in by complement dependent cytotoxicity (CDC), Antibody dependent cell-mediated cytotoxicity (ADCC) assays of cells that express a target antigen, such as CLL-1. Exemplary cells that express CLL-1 include cell lines that express heterologous, recombinant CLL-1 (e.g., human CLL-1); human AML cell lines such as HL60, THP1, TF1-alpha, U937, and OCI AML-5 (the first four of which are available from ATCC); primary cells from one or more AML patients (e.g., PBMC or engrafted tumor cells); human CML cell lines such as K562 and KU812 (available from ATCC); and primary cells from one or more CML or MDS patients.

An antibody is described as having CDC activity and mediating CDC if it results in complement dependent killing of cells that express the antibody target. CDC assays are known in the art, and are described, e.g., in Gazzano-Santoro et al. (1997) J. Immunol. Methods 202:163; Idusogie et al. (2000) J. Immunol. 164:4178; and in Example 6 below. CDC kits and services are commercially available, e.g. from GeneScript® and Cell Technology Inc.

In brief, the assay is typically carried out in vitro, and includes antibody binding to a cell expressing the antibody target on its surface. Complement components, including C1q,22 which binds to the Ch region of the antibody, are added. The complement components then interact to kill the targeted cell. CDC is measured after a period of incubation of generally between 4 and 24 hours, for example, by determining the release of intracellular enzyme or granules known to be present in the targeted cell, by comparing the starting and ending target cell population, etc.

An antibody is described as having ADCC activity and mediating ADCC if it results in killing of antibody-bound cells (e.g., CLL-1 expressing cells) by effector cells. Effector cells are typically natural killer cells, but can also be macrophages, neutrophils, or eosinophils.

Genetically engineered effector cell lines have also been developed for use in ADCC assays (see, e.g., Schnueriger et al. (2011) Mol. Immunol. 48:1512). ADCC assays are known in the art, and are described, e.g., in Perussia and Loza (2000)Methods in Mol. Biol. 121:179; Bretaudeau and Bonnaudet (2011) BMC Proceedings 5(Suppl 8):P63; ADCC kits and services are commercially available, e.g. from GeneScript® and Promega®, and in the Example below.

In brief, the assay is typically carried out in vitro, and includes antibody binding to a cell expressing the antibody target on its surface. Effector cells are added that recognize antibody-bound cells, typically through an Fc receptor such as CD 16. The effector cells kill the antibody-bound cell, e.g., by releasing cytotoxins that cause apoptosis. Cell death is detected by release of a detectable element within the target cells (e.g., Cr51) or by detection of an element involved in the cell mediated toxicity (e.g., activation of NFAT signaling in effector cells).

An antibody is described as having antibody-drug conjugate (ADC) activity (or mediating ADC) if the antibody, when conjugated with a cytotoxic agent (drug), results in killing (inhibiting survival) a cell that expresses the target of the antibody, in this case, CLL-1. Appropriate cytotoxic agents are known in the art, e.g., saporin, doxorubicin, daunomycin, vinca-alkaloids, taxoids, tubulin agents (e.g., Maytansin, auristatin), and DNA agents (e.g., calicheamicin, duocarmycin, pyrrolobenzodiazepine dimers), etc. ADC assays are known in the art, e.g., as described in Gerber et al. (2009) 3:247, and in the Examples below.

C. Diagnostic Applications

The cysteine substituted immunoglobulin conjugates can thus be used for in vitro and in vivo diagnostic assays to detect cancer cells. This includes antibodies specific to CLL-1 described herein specifically bind CLL-1-expressing cells (“CLL-1 antibody”—for this section only) for detection of CLL-1-expressing cells (e.g., AML cells and AML CSCs). For example, a sample (e.g., blood sample or tissue biopsy) can be obtained from a patient and contacted with a CLL-1 antibody, and the presence of a CLL-1-expressing cell in the patient sample can be determined by detecting antibody binding. Antibody binding can be detected directly (e.g., where the antibody itself is labeled) or by using a second detection agent, such as a secondary antibody. The detectable label can be associated with an antibody of the disclosure, either directly, or indirectly, e.g., via a chelator or linker.

In some embodiments, the CLL-1 antibody is contacted with a biological sample from an individual having or suspected of having a CLL-1 associated disorder, and antibody binding to a cell in the sample is determined, wherein higher or lower than normal antibody binding indicates that the individual has a CLL-1 associated disorder. In some embodiments, the biological sample is a blood sample or blood fraction (e.g., serum, plasma, platelets, red blood cells, white blood cells, PBMCs). In some embodiments, the biological sample is a tissue sample (biopsy), e.g., from a suspected tumor site, or from a tissue that is known to be affected, e.g., to determine the boundaries of a known tumor.

Biopsies are typically performed to obtain samples from tissues, i.e., non-fluid cell types. The biopsy technique applied will depend on the tissue type to be evaluated (e.g., breast, skin, colon, prostate, kidney, lung, bladder, lymph node, liver, bone marrow, airway or lung). In the case of a cancer the technique will also depend on the size and type of the tumor (e.g., solid, suspended, or blood), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

Any method of detecting antibody binding to a cell in a sample can be used for the present diagnostic assays. Methods of detecting antibody binding are well known in the art, e.g., flow cytometry, fluorescent microscopy, ELISAs, etc. In some embodiments, the method comprises preparing the biological sample for detection prior to the determining step. For example, a subpopulation of cells (e.g., white blood cells, CD34+ cells, CD45+ cells, etc.) can be separated from the rest of the sample from the individual (e.g., other blood components) or cells in a tissue can be suspended for easier detection.

In some embodiments, the percentage of CLL-1-expressing cells in the sample is determined and compared to a control, e.g., a sample from an individual or group of individuals that are known to have a CLL-1 associated disorder (positive control) or from an individual or group of individuals that are known not to have a CLL-1 associated disorder (normal, healthy, non-disease, or negative control). In some embodiments, the control is a standard range of CLL-1 expression established for a given tissue. A higher or lower than normal percentage of CLL-1 expressing cells, or higher or lower expression level, indicates that the individual has a CLL-1 associated disorder.

In some embodiments, a labeled CLL-1 antibody can be provided (administered) to an individual to determine the applicability of an intended therapy. For example, a labeled antibody may be used to detect CLL-1 density within a diseased area, where the density is typically high relative to non-diseased tissue. A labeled antibody can also indicate that the diseased area is accessible for therapy. Patients can thus be selected for therapy based on imaging results. Anatomical characterization, such as determining the precise boundaries of a cancer, can be accomplished using standard imaging techniques (e.g., CT scanning, MRI, PET scanning, etc.).

In some embodiments, labeled CLL-1 antibodies as described herein can be further associated with a therapeutic compound, e.g., to form a “theranostic” composition. For example, an CLL-1 antibody can be linked (directly or indirectly) to both a detectable label and a therapeutic agent, e.g., a cytotoxic agent to kill CLL-1-expressing cancer cells. In some embodiments, a labeled CLL-1 antibody is used for diagnosis and/or localization of a CLL-1 expressing cancer cell, and the CLL-1 expressing cancer cell is then targeted with a separate therapeutic CLL-1 specific antibody. In some embodiments, the diagnostic CLL-1 specific antibody is one that is not internalized into CLL-1-expressing cells at a high rate or percentage. In some embodiments, the therapeutic CLL-1 antibody is internalized into CLL-1-expressing cells at a high rate or percentage.

1. Labels

A diagnostic agent comprising an antibody capable of binding a target of interest can include any diagnostic agent known in the art, as provided, for example, in the following references: Armstrong et al., Diagnostic Imaging, 5th Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). A diagnostic agent can be detected by a variety of ways, including as an agent providing and/or enhancing a detectable signal. Detectable signals include, but are not limited to, gamma-emitting, radioactive, echogenic, optical, fluorescent, absorptive, magnetic, or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like. The terms “detectable agent,” “detectable moiety,” “label,” “imaging agent,” and like terms are used synonymously herein.

In some embodiments, the label can include optical agents such as fluorescent agents, phosphorescent agents, chemiluminescent agents, and the like. Numerous agents (e.g., dyes, probes, labels, or indicators) are known in the art and can be used in the present disclosure. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives. Fluorescent dyes are discussed, for example, in U.S. Pat. No. 4,452,720, U.S. Pat. No. 5,227,487, and U.S. Pat. No. 5,543,295.

The label can also be a radioisotope, e.g., radionuclides that emit gamma rays, positrons, beta and alpha particles, and X-rays. Suitable radionuclides include but are not limited to 225Ac, 72As, 211At, 11B, 128Ba, 212Bi, 75Br, 77Br, 14C, 109Cd, 2Cu, 64Cu, 67Cu, 18F, 67Ga, 68Ga, 3H, 166Ho, 123I, 124I, 125I, 130I, 131I, 111In, 177Lu, 13N, 15O, 32P, 33P, 212Pb, 103Pd,186Re, 188Re, 47Sc, 153Sm, 89Sr, 99mTc, 88Y and 90Y. In some embodiments, radioactive agents can include 111In-DTPA,99mTc(C0)3-DTPA, 99mTc(CO)3-ENPy2, 62/64/67Cu-TETA, 99mTc(CO)3-IDA, and 99mTc(CO)3-triamines (cyclic or linear). In some embodiments, the agents can include DOTA and its various analogs with 111In, 177Lu, 153Sm, 62/64/67Cu, or67/68Ga. In some embodiments, a nanoparticle can be labeled by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging. 33:1196-1205 (2006); Mougin-Degraef, M. et al., Int'l. J. Pharmaceutics 344:110-117 (2007).

In some embodiments, the diagnostic agent can be associated with a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Secondary binding ligands include, e.g., biotin and avidin or streptavidin compounds as known in the art.

In some embodiments, the labeled antibody can be further associated to a composition that improves stability in vivo, e.g. PEG or a nanoparticle such as a liposome, as described in more detail below.

2. Methods of Labeling

Techniques for conjugating detectable and therapeutic agents to antibodies are well known (see, e.g., Amon et al., “Monoclonal Antibodies For Immunotargeting Of Drugs In Cancer Therapy”, in Monoclonal Antibodies And Cancer Therapy. Reisfeld et al. (eds.), pp. 243-56 (Alan R. Liss, Inc, 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera, et al. (eds.), pp. 475-506 (1985); and Thorpe et al., “The Preparation And. Cytotoxic Properties Of Antibody-Toxin Conjugates”, Immunol. Rev., 62:119-58 (1982)).

Typically, the antibody is attached to detectable moiety in an area that does not interfere with binding to the epitope. Thus in some cases, the detectable moiety is attached to the constant region, or outside the CDRs in the variable region. One of skill in the art will recognize that the detectable moiety can be located elsewhere on the antibody, and the position of the detectable moiety can be adjusted accordingly. In some embodiments, the ability of the antibody to associate with the epitope is compared before and after attachment to the detectable moiety to ensure that the attachment does not unduly disrupt binding.

In some embodiments, the antibody can be associated with an additional targeting moiety. For example, an antibody fragment, peptide, or aptamer that binds a different site on the target molecule or target cell can be conjugated to the antibody to optimize target binding, e.g., to a cancer cell.

C. Therapeutic Applications

CLL-1-expressing cells such as AML cells can be targeted using the cysteine substituted CLL-1 ADC antibodies described herein (“CLL-1 Antibodies”—for this section only). CLL-1 expression is elevated on AML cells and CSCs (e.g., AML CSCs). CLL-1 is not significantly expressed on normal CD34+ hematopoietic stem cells (HSCs), thus CSCs can be distinguished from HSCs using the present CLL-1 antibodies. High affinity CLL-1 antibodies that recognize a CLL-1 epitope common to AML cells, and thus able to universally bind to AML cells, is particularly valuable, as AML has a very high rate of recurrence. As noted above, a therapeutic composition comprising CLL-1 antibody can further include a detectable label to form a theranostic composition, e.g., for detection and localization of CLL-1 expressing cells, and monitoring of therapeutic effect. Sequences of antibodies that bind CLL-1 are described in U.S. SN 62/259,100, filed Nov. 24, 2015 (Jiang et al., “Humanized Anti-”) and in US 2013/0295118 published Nov. 7, 2013 (Jiang et al., “Antibodies Specific For CLL-1), incorporated by reference in their entirety.

Antibodies that bind targets other than CLL-1 can also be used in the cysteine-substituted antibody and antibody conjugates described herein. In some embodiments, the antibody targets can be selected from GPR114, CLL-1, ILIRAP, TIM-3,CD19, CD20, CD22, ROR1, mesothelin, CD33, CD123/IL3Ra, c-Met, PSMA, prostatic acid phosphatase (PAP), CEA, CA-125, Muc-1, AFP, Glycolipid F77, EGFRvIII, GD-2, NY-ESO-1 TCR, tyrosinase, TRPI/gp75, gp100/pmel-17, Melan-A/MART-1, Her2/neu, WT1, , EphA3, telomerase, HPV E6, HPV E7, EBNA1, BAGE, GAGE and MAGE A3 TCRSLITRK6, ENPP3, Nectin-4, CD27, SLC44A4, CAIX, Cripto, CD30, MUC16, GPNMB, BCMA, Trop-2, Tissue Factor (TF), CanAg, EGFR, αv-integrin, CD37, Folate Receptor, CD138, CEACAM5, CD56, CD70, CD74, GCC, 5T4, CD79b, Steap1, Napi2b, Lewis Y Antigen, LIV, c-RET, DLL3, EFNA4, or Endosialin/CD248.

As demonstrated herein, the present CLL-1 antibodies can inhibit cancer cell growth (proliferation and/or engraftment) and thus can be considered chemotherapeutic agents alone. The following disclosure provides examples of chemotherapeutic and cytotoxic agents that can be linked to CLL-1 antibody for additional effect on CLL-1-expressing cells.

A chemotherapeutic (anti-cancer) agent can be any agent capable of reducing cancer growth, interfering with cancer cell replication, directly or indirectly killing cancer cells, reducing metastasis, reducing tumor blood supply, etc. Chemotherapeutic agents thus include cytotoxic agents. Cytotoxic agents include but are not limited to saporin, taxanes, vinca alkaloids, anthracycline, and platinum-based agents. Classes of chemotherapeutic agents include but are not limited to alkylating agents, antimetabolites, e.g., methotrexate, plant alkaloids, e.g., vincristine, and antibiotics, e.g., doxorubicin as well as miscellaneous drugs that do not fall in to a particular class such as hydroxyurea. Platinum-based drugs, exemplified by cisplatin and oxaliplatin, represent a major class of chemotherapeutics. These drugs bind to DNA and interfere with replication. Taxanes, exemplified by taxol, represent another major class of chemotherapeutics. These compounds act by interfering with cytoskeletal and spindle formation to inhibit cell division, and thereby prevent growth of rapidly dividing cancer cells. Other chemotherapeutic drugs include hormonal therapy. Drug moieties can include cytotoxic agents, such as a monomeric or dimeric benzodiazepine derivative (see, e.g., U.S. patent application Ser. No. 15/048,865, which is incorporated for reference), dolastatins, auristatins, maytansinoid, dolastatin, tubulysin, cryptophycin, pyrrolobenzodiazepine (PBD) dimer, indolinobenzodiazepine dimer, isoquinolidinobenzodiazepine dimer (including but not limited to D202 as described below), alpha-amanitin, trichothene, SN-38, duocarmycin, CC1065, calicheamincin, an enediyne antibioatic, taxane, doxorubicin derivatives, anthracycline and stereoisomers, azanofide, isosteres, analogs or derivatives thereof.

More than one therapeutic agent can be combined, either in the same composition, or in separate compositions. The therapeutic agent(s) can also be combined with additional therapeutic agents as appropriate for the particular individual. Common therapeutic agents provided to cancer patients include medications to address pain, nausea, anemia, infection, inflammation, and other symptoms commonly experienced by cancer patients.

Antibodies can be attached to a therapeutic agent, detectable agent, or nanocarrier using a variety of known cross-linking agents. Methods for covalent or non-covalent attachment of polypeptides are well known in the art. Such methods may include, but are not limited to, use of chemical cross-linkers, photoactivated cross-linkers and/or bifunctional cross-linking reagents. Exemplary methods for cross-linking molecules are disclosed in U.S. Pat. No. 5,603,872 and U.S. Pat. No. 5,401,511. Non-limiting examples of cross-linking reagents include glutaraldehyde, bifunctional oxirane, ethylene glycol diglycidyl ether, carbodiimides such as 1-ethyl-3-(3-dimethylaminopropyl) carbodiimide or dicyclohexylcarbodiimide, bisimidates, dinitrobenzene, N-hydroxysuccinimide ester of suberic acid, disuccinimidyl tartarate, dimethyl-3,3′-dithio-bispropionimidate, azidoglyoxal, N-succinimidyl-3-(2-pyridyldithio)propionate and 4-(bromoadminoethyl)-2-nitrophenylazide.

In some embodiments, the CLL-1 antibody is associated with a nanocarrier. For antibodies conjugated to nanocarriers (e.g., liposomes), a certain number of antibodies will be present on the surface, i.e., at a given surface density. In some embodiments, the nanocarrier will have at least 5 antibodies per nanocarrier, e.g., at least 10, 30, 40, 50, 75, 100 or higher antibodies per nanocarrier. One of skill in the art will understand that surface density represents an average range, as the number of antibodies per nanocarrier will not be absolutely uniform for all members of the population.

Nanocarriers include vesicles such as liposomes and micelles, as well as polymeric nanoparticles, etc. Nanocarriers are useful for delivery of therapeutic and diagnostic agents, but can be particularly useful for shielding cytotoxic agents used to treat cancer. The nanocarrier can comprise lipids (e.g., phospholipids), hydrophilic polymers, hydrophobic polymers, amphipathic compounds, cross-linked polymers, and a polymeric matrix (see, e.g., WO2009/110939). Depending on the application, the nanocarrier can be designed to have a particular size, half-life, shelf life, and leakage rate.

Preparation of nanocarriers, such as an antibody targeted liposome, polymeric nanoparticle, or extended shelf-life liposome, is described, e.g., in U.S. Pat. Nos. 6,465,188, 7,122,202, 7,462,603 and 7,550,441.

In some embodiments, the antibody is linked to a stabilizing moiety such as PEG, or a liposome or other nanocarrier. U.S. Pat. Nos. 4,732,863 and 7,892,554 and Chattopadhyay et al. (2010) Mol Pharm 7:2194 describe methods for attaching the selected antibody to PEG, PEG derivatives, and nanoparticles (e.g., liposomes). Liposomes containing phosphatidyl-ethanolamine (PE) can be prepared by established procedures as described herein. The inclusion of PE provides an active functional site on the liposomal surface for attachment.

The antibody conjugate can also be formulated to provide more than one active compound, e.g., additional chemotherapeutic or cytotoxic agents, cytokines, or growth inhibitory agents. The active ingredients may also prepared as sustained-release preparations (e.g., semi-permeable matrices of solid hydrophobic polymers (e.g., polyesters, hydrogels (for example, poly (2-hydroxyethyl-methacrylate), or poly (vinylalcohol)), polylactides. The antibodies and immunoconjugates can be entrapped in a nanoparticle prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nano-particles and nanocapsules) or in macroemulsions.

The CLL-1 antibodies described herein can kill CLL-1-expressing cells alone, or in combination with a cytotoxic agent. In some embodiments, the method of treatment comprises administering to an individual an effective amount of a therapeutic CLL-1 antibody or CLL-1 antibody conjugate, e.g., a CLL-1 antibody attached to a therapeutic agent. In some embodiments, the individual has been diagnosed with cancer, e.g., AML. In some embodiments, the individual is receiving or has received cancer therapy, e.g., surgery, radiotherapy, or chemotherapy. In some embodiments, the individual has been diagnosed, but the cancer is in remission.

In some embodiments, the method further comprises monitoring the individual for progression of the cancer. In some embodiments, the dose of the CLL-1 antibody or CLL-1 antibody conjugate for each administration is determined based on the therapeutic progress of the individual, e.g., where a higher dose of chemotherapeutic is administered if the individual is not responding sufficiently to therapy.

In some embodiments, the disclosure can include an antibody or antibody-targeted composition and a physiologically (i.e., pharmaceutically) acceptable carrier. The term “carrier” refers to a typically inert substance used as a diluent or vehicle for a diagnostic or therapeutic agent. The term also encompasses a typically inert substance that imparts cohesive qualities to the composition. Physiologically acceptable carriers can be liquid, e.g., physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present disclosure (See, e.g., Remington's Pharmaceutical Sciences, 17th ed., 1989).

The compositions of the present disclosure may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized antibody compositions.

Dosage forms can be prepared for mucosal (e.g., nasal, sublingual, vaginal, buccal, or rectal), parenteral (e.g., subcutaneous, intravenous, intramuscular, or intraarterial injection, either bolus or infusion), oral, or transdermal administration to a patient. Examples of dosage forms include, but are not limited to: dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Injectable (e.g., intravenous) compositions can comprise a solution of the antibody or antibody-targeted composition suspended in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. Often, normal buffered saline (135-150 mM NaCl) will be used. The compositions can contain pharmaceutically acceptable auxiliary substances to approximate physiological conditions, such as pH adjusting and buffering agents, tonicity adjusting agents, wetting agents, e.g., sodium acetate, sodium lactate, sodium chloride, potassium chloride, calcium chloride, sorbitan monolaurate, triethanolamine oleate, etc. In some embodiments, the antibody-targeted composition can be formulated in a kit for intravenous administration.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The pharmaceutical preparation can be packaged or prepared in unit dosage form. In such form, the preparation is subdivided into unit doses containing appropriate quantities of the active component, e.g., according to the dose of the therapeutic agent or concentration of antibody. The unit dosage form can be a packaged preparation, the package containing discrete quantities of preparation, in unit-dose or multi-dose sealed containers, such as ampoules and vials. The composition can, if desired, also contain other compatible therapeutic agents.

The antibody (or antibody-targeted composition) can be administered by injection or infusion through any suitable route including but not limited to intravenous, subcutaneous, intramuscular or intraperitoneal routes. An example of administration of a pharmaceutical composition includes storing the antibody at 10 mg/mL in sterile isotonic aqueous saline solution for injection at 4° C., and diluting it in either 100 mL or 200 mL 0.9% sodium chloride for injection prior to administration to the patient. The antibody is administered by intravenous infusion over the course of 1 hour at a dose of between 0.2 and 10 mg/kg. In other embodiments, the antibody is administered by intravenous infusion over a period of between 15 minutes and 2 hours. In still other embodiments, the administration procedure is via sub-cutaneous bolus injection.

The dose of antibody is chosen in order to provide effective therapy for the patient and is in the range of less than 0.1 mg/kg body weight to about 25 mg/kg body weight or in the range 1 mg-2 g per patient. In some cases, the dose is in the range 1-100 mg/kg, or approximately 50 mg-8000 mg/patient. The dose may be repeated at an appropriate frequency which may be in the range once per day to once every three months, depending on the pharmacokinetics of the antibody (e.g., half-life of the antibody in the circulation) and the pharmacodynamic response (e.g., the duration of the therapeutic effect of the antibody). In some embodiments, the in vivo half-life of between about 7 and about 25 days and antibody dosing is repeated between once per week and once every 3 months.

Administration can be periodic. Depending on the route of administration, the dose can be administered, e.g., once every 1, 3, 5, 7, 10, 14, 21, or 28 days or longer (e.g., once every 2, 3, 4, or 6 months). In some cases, administration is more frequent, e.g., 2 or 3 times per day. The patient can be monitored to adjust the dosage and frequency of administration depending on therapeutic progress and any adverse side effects, as will be recognized by one of skill in the art.

Thus in some embodiments, additional administration is dependent on patient progress, e.g., the patient is monitored between administrations. For example, after the first administration or round of administrations, the patient can be monitored for rate of tumor growth, recurrence (e.g., in the case of a post-surgical patient), or general disease-related symptoms such as weakness, pain, nausea, etc.

For the treatment of cancer, an antibody or antibody-targeted composition (e.g., including a therapeutic and/or diagnostic agent) can be administered at the initial dosage of about 0.001 mg/kg to about 1000 mg/kg daily and adjusted over time. A daily dose range of about 0.01 mg/kg to about 500 mg/kg, or about 0.1 mg/kg to about 200 mg/kg, or about 1 mg/kg to about 100 mg/kg, about 5 to about 10 mg/kg, or about 10 mg/kg to about 50 mg/kg, can be used. The in vivo xenograft results described herein indicate that a dose between 5-20 mg antibody/kg body weight is effective for dramatic reduction of tumor growth.

The dosage is varied depending upon the requirements of the patient, the severity of the condition being treated, and the targeted composition being employed. For example, dosages can be empirically determined considering the type and stage of cancer diagnosed in a particular patient. The dose administered to a patient, in the context of the present disclosure, should be sufficient to affect a beneficial therapeutic response in the patient over time. The size of the dose will also be determined by the existence, nature, and extent of any adverse side-effects that accompany the administration of a particular targeted composition in a particular patient, as will be recognized by the skilled practitioner.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entireties for all purposes.

All publications and patent applications mentioned in this specification are herein expressly incorporated by reference in their entirety to the same extent as if each individual publication or patent application were specifically and individually indicated to be incorporated by reference.

From the foregoing it will be appreciated that, although specific embodiments of the Invention have been described herein for purposes of illustration, various modifications may be made without deviating from the spirit and scope of the invention. Accordingly, the invention is not limited except as by the appended claims.

The following examples are offered by way of illustration and not by way of limitation.

### VI. EXAMPLES

A cysteine residue was engineered at selected position (EU numbering) of CLL-1 antibody (HuM31) heavy chain to produce corresponding CYSMAB variant using QuickChange II Site-Directed Mutagenesis Kit (Agilent). Authenticity of the cysteine substitution was verified by DNA sequencing. CYSMAB light and heavy chain construct were transiently transfected into HEK-293 cells. Expressed CYSMAB variant was purified using MabSelectsuRe beads and further characterized with various CLL-1 functional assays.

### Example 1

### Conjugation

To demonstrate conjugation at selected residues of the heavy chain constant region, antibody-fluorophore conjugates were created. The following procedure was used: Purified HuM31 or CYSMAB variants (1.5 mg each) were dialyzed against PBS overnight at 4° C. Antibodies were incubated with 200 μL of MabSelectsuRe beads at room temperature for 1 hour. After washing beads three times with 2 mL PBS each time, antibodies were reduced in 2 mM DTT at RT overnight in 150 mM NaCl-50mM Tris, pH 8.0 buffer. Beads were washed three times then antibodies were re-oxidized in 1 mM Dehydroascorbic acid (DHAA) at room temperature for three hours. Antibodies were washed three times and conjugated with 10 molar excess Alexa488-C5-maleimide at room temperature for two hours. Beads were washed three times and Alexa488 conjugated antibodies were eluted with 500 μL of 0.1M Glycine, pH 2.7. The antibody concentration and Alexa488 conjugation efficiency (number of Alexa488 per antibody) was determined by using NanoDrop 2000.

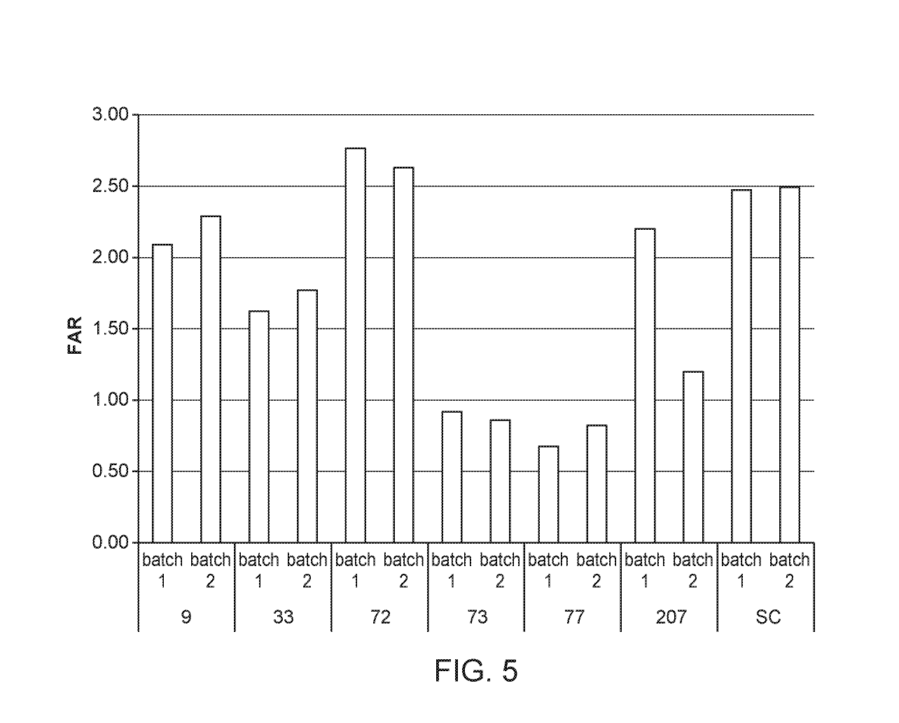

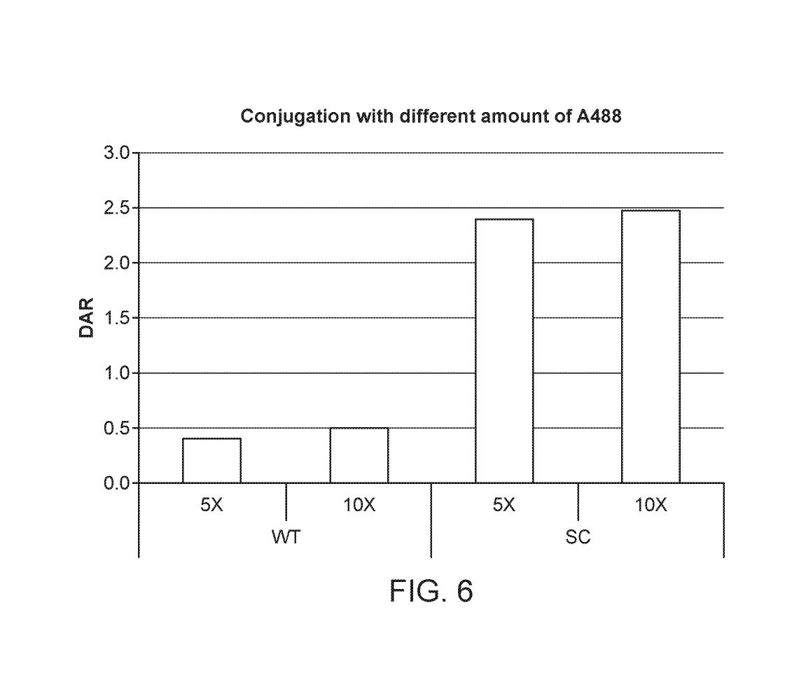

To demonstrate that the conjugation was not variable with the date or amount of fluorophore, the conjugation procedure was repeated on different dates, and with different concentrations. The results (FIG. 5 and FIG. 6, respectively) show that the neither the conjugation ratio (e.g., DAR, FAR) vary appreciably in the procedure.

The results of the conjugation, including amino acid residue and fluorophore-to-antibody ratio (“FAR”) are reported in FIGS. 7-9.

FIG. 7 shows of 45 total conjugations, 21 (47%) displayed high conjugation (>2), 7 (16%) medium (1-2) and 17 (38%) low (<1).

FIG. 8 shows of 20 total conjugates, 10 (50%) display high conjugation (>2), 1 (5%) medium (1-2) and 9 (45%) low (<1).

### Example 2

### Specificity ELISA

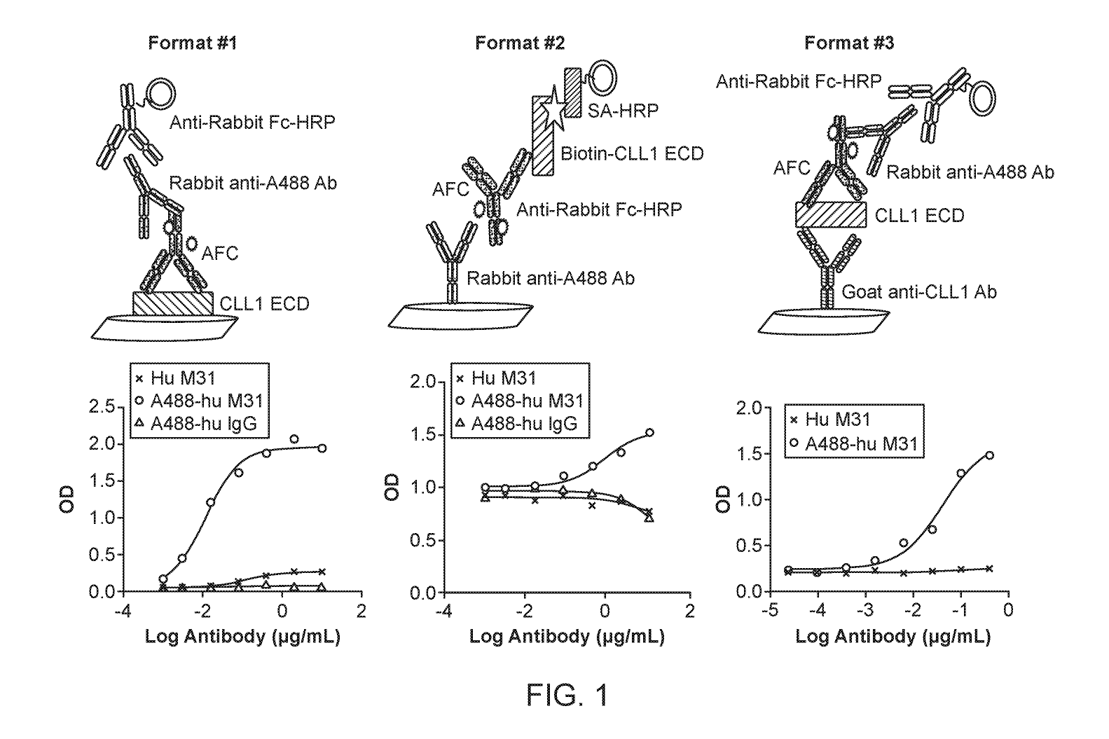

FIG. 1: An ELISA assay specifically to detect Alexa 488 (A488) conjugated to HuM31 (HuM31-A488 AFC) was developed. Three different formats of ELISA were designed to detect the A488 conjugated to HuM31 in human plasma. Unconjugated HuM31, HuM31-A488 and IgG-A488 were tested in these three ELISA methods. The results showed ELISA format #1 has best signal to noise ratio. And format #2 and #3 showed higher background binding. Format #1 ELISA was moved forward to detect HuM31-A488.

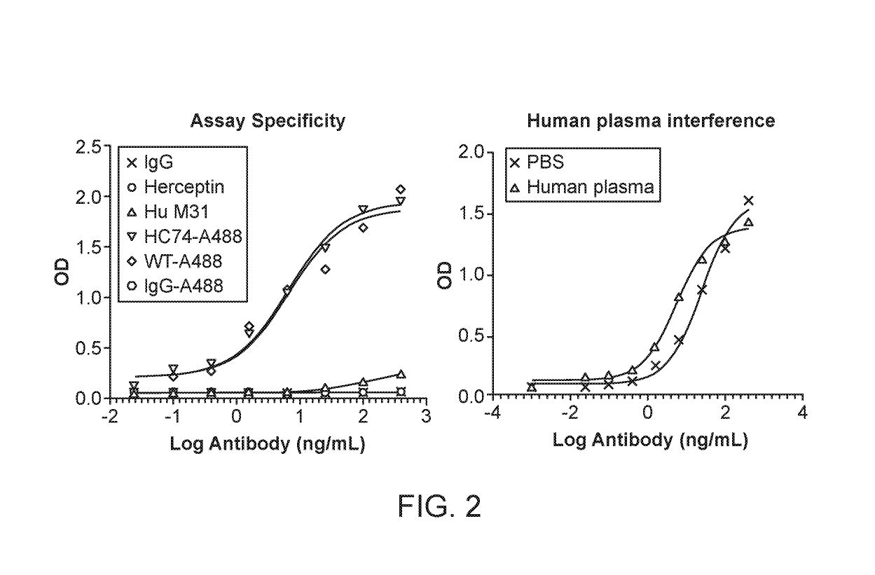

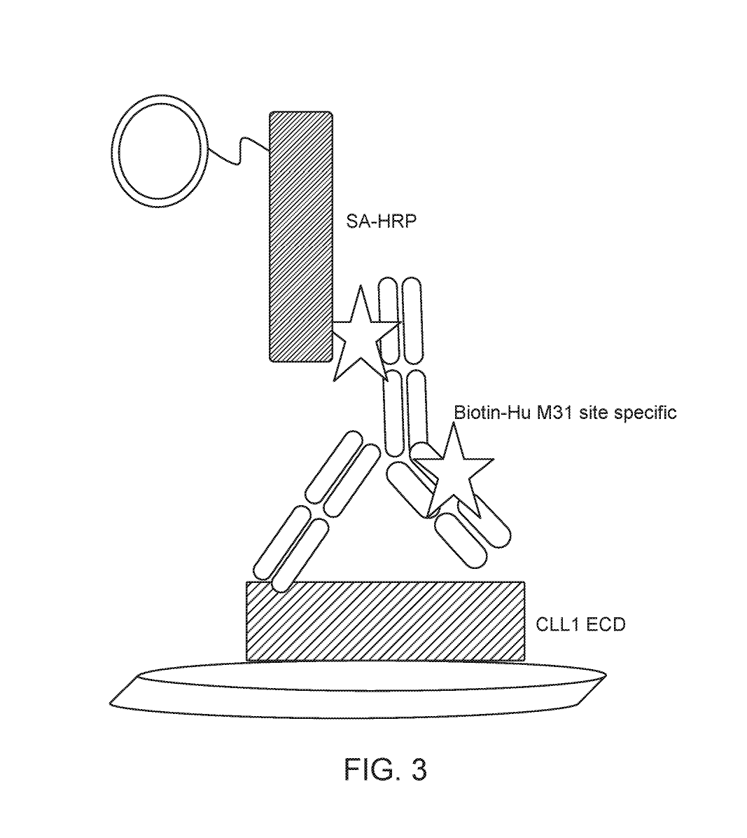

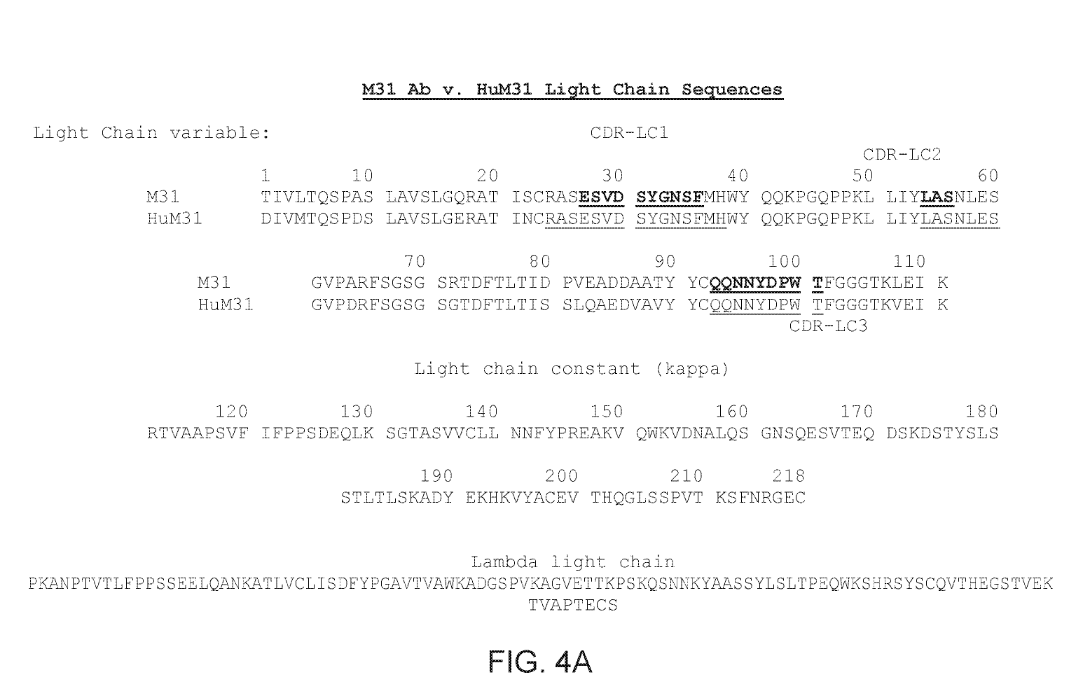

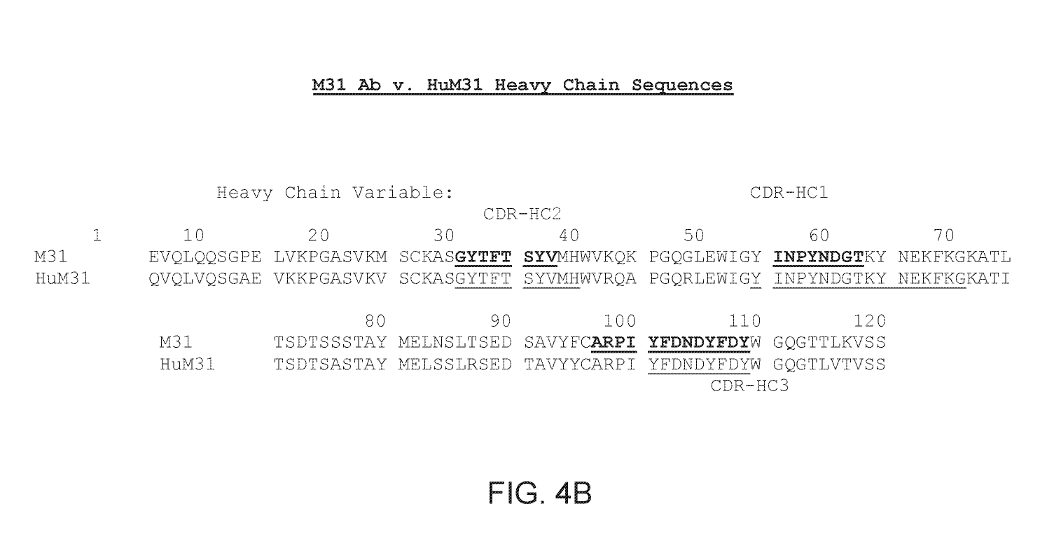

FIG. 2a: Specificity of the ELISA assay. Format #1 ELISA method was used to specifically detect HuM31-A488 and site-specific CYSMAB-A488 conjugates instead of control samples (relative to IgG (Isotype control), IgG-A488 AFC, trastuzumab and unconjugated HuM31) used. The result demonstrated that this ELISA method only detected HuM31-A488 and site-specific CYSMAB-A488 conjugates. In contrast controls: isotype human IgG, trastuzumab, HuM31 or IgG-A488 conjugates showed no binding.

FIG. 2b: Testing human plasma interference in the ELISA assay. Under the optimal ELISA conditions, the presence of 1% human plasma only marginally enhanced the binding of HuM31-A488 conjugate to anti-A488 antibody. Therefore the ELISA method can be used to analyze antibody conjugate samples that had prior exposure to human plasma.

### Example 3

### Stability of HuM31-A488 Conjugate (AFC)

The stability in human plasma of the AFCs of the disclosure was tested by incubation in human plasma. AFC (50 μg/mL) was spiked into pooled human plasma or 0.5% BSA in PBS. Each sample was then incubated at 37° C. with 5% CO₂, and then transferred to −80° C. at 0, 24, 48, 72 and 96 hour time points. The samples were diluted 1:5000 in sample Diluent (PBS buffer containing 0.5% BSA, 0.05% Tween 20, 5 mM EDTA, 0.35M NaCl, 0.25% CHAPS and 0.2% BGG). The samples at various time points were then assayed by the ELISA.

ELISA procedure: CLL-1 extra cellular domain protein in PBS (1 μg/mL) was coated on 96-well plate and incubated overnight at 4° C. Plate then washed three times with 0.1% Tween 20 PBS followed by 1 hour of blocking with 1% BSA in 0.1% Tween 20 PBS at room temperature. After six times washes with 0.1% Tween 20 in PBS, serial diluted Alexa488 conjugated human M31 and its controls were added to the plate and incubated for 1 hour at room temperature. Then plate was washed with 0.1% Tween 20 in PBS. Rabbit anti-Alexa 488 secondary antibody (1 μg/mL) was added to the plate and incubated for 1 hour at RT. After six times washes with 0.1% Tween 20 PBS plate was detected by HRP conjugated goat anti-rabbit Fc polyclonal antibody at 1:50,000 dilution. The percent (%) values were then evaluated by comparing the OD value of each time point to time 0.

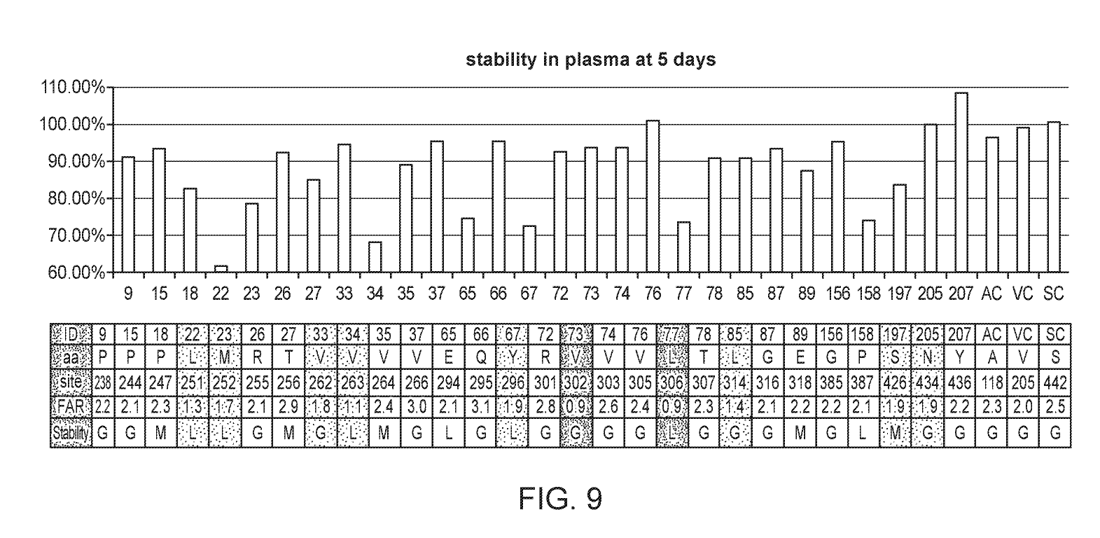

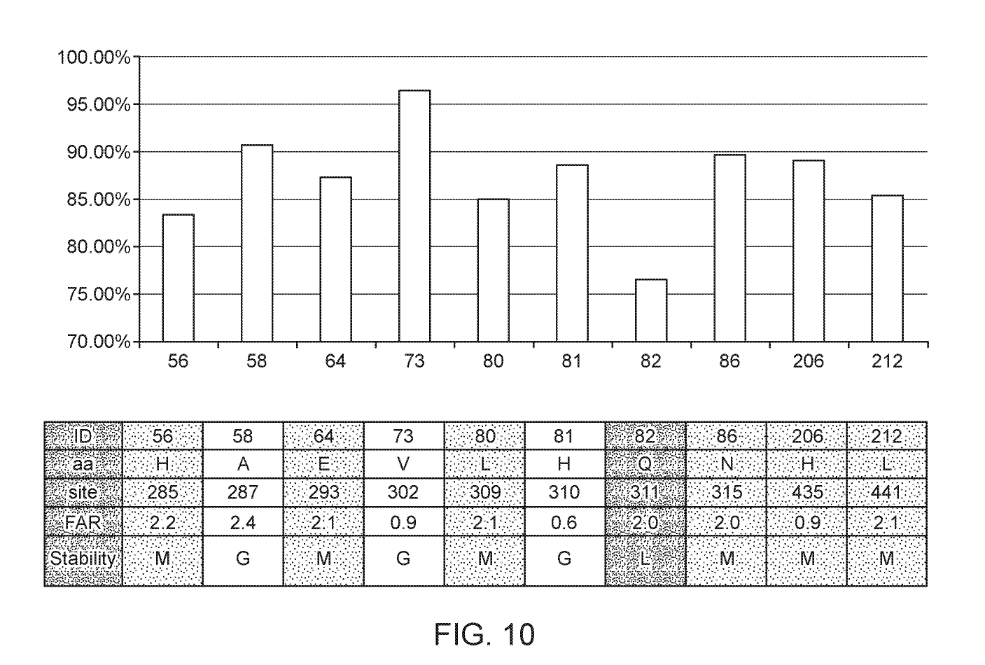

The stability of tested samples after 5 days are shown in FIGS. 9 and 10:

FIGS. 9 and 10 show that samples 58, 64, 73, 81, 86, and 206 have stability >85% after 5 days of incubation.

### Example 4

### Stability of HuM31-Biotin Conjugates

HuM31-Biotin conjugates were generated by conjugating CYSMABs with HPDP-biotin and BMCC-biotin.

Purified human M31 or CYSMAB variants (1.5 mg each) were dialyzed against PBS overnight at 4° C. Antibodies were incubated with 200 μL of MabSelectsuRe beads at room temperature for 1 hour. After washing beads three times with 2 mL PBS each time, antibodies were reduced in 2 mM DTT at RT overnight in 150 mM NaCl-50 mM Tris, pH 8.0 buffer. Beads were washed three times then antibodies were re-oxidized in 1 mM DHAA at room temperature for three hours. Antibodies were washed three times and conjugated with 10 molar excess HPDP-biotin or BMCC biotin at room temperature for two hours. Beads were washed three times and biotin conjugated antibodies were eluted with 500 μL of 0.1M Glycine, pH 2.7. The antibody concentration and biotin conjugation efficiency was determined by using NanoDrop 2000 and ELISA based assay, respectively.

An ELISA assay was developed to determine stability of ADCs in human plasma.: CLL-1 extra cellular domain (1 μg/mL) in PBS was coated on 96-well plate and incubated overnight at 4° C. ELISA plate was washed three times with washing buffer (0.1% Tween 20 in PBS) followed by 1 hour of blocking with 1% BSA in 0.1% Tween 20 in PBS at room temperature. After washing the plate six times with washing buffer, serially diluted HuM31-Biotin and its corresponding control samples were added to the plate and incubated for 1 hour at room temperature. The plate was washed six time with washing buffer followed by detection using streptavidin-HRP conjugate (used at 1:100,000 dilution).

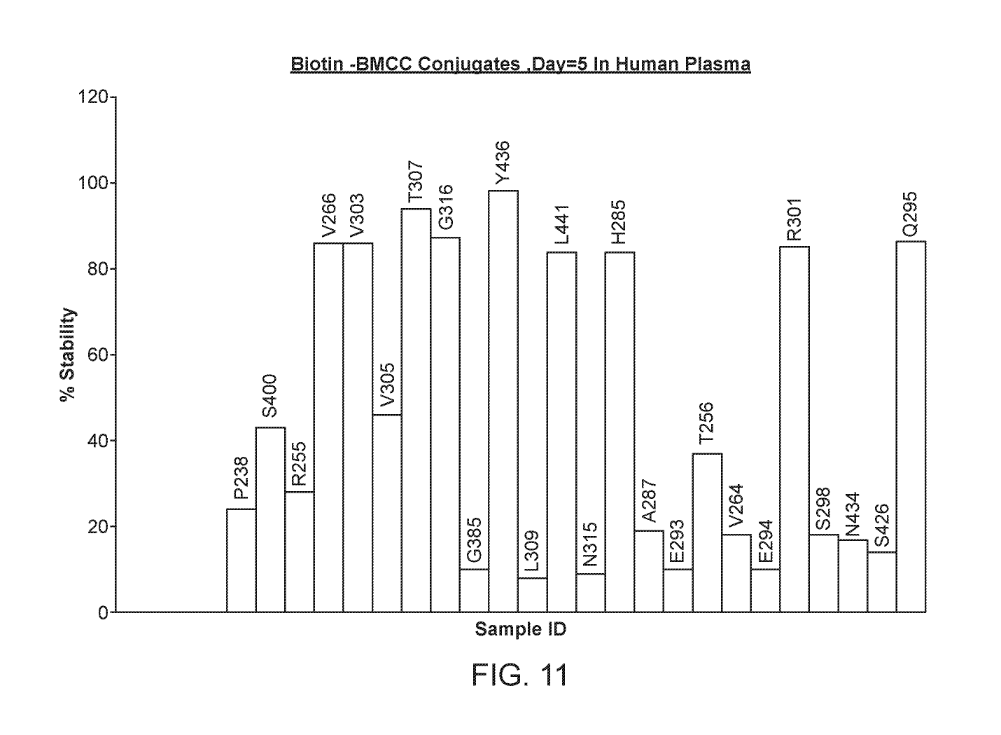

The stability of the HPDP and BMCC linked antibody conjugates after day 5 are reported in FIG. 11.

### The Stability of Biotin-BMCC Conjugate Samples in Human Plasma:

FIG. 11 shows that samples V266, V303, T307, G316, Y436, L441, H285, R301, Q295 have stability >80% after 5 days of incubation.

### Example 5

### Affinity Testing

The binding affinity for the cysteine-substituted CLL-1 CYSMABs can be tested for comparative binding affinity to their naked, unconjugated counterparts. Briefly, biotinylated CLL-1 (25 μg/mL) is loaded onto streptavidin sensor tips for 2 hours at 22° C. Ab-Ag dissociation curves were generated at three different concentrations for each antibody with either Fortebio or BIAcore analysis (10, 30, and 90 μg/mL) using a global 1:1 curve fitting.

### Example 6

### Binding to AML Cell Lines and AML Patient Samples

The cysteine-substituted CYSMABs can be tested for comparative binding to recombinant 293 cells expressing human CLL-1, and two AML cell lines, HL60 and OCI AML-5. The percentage of live cells with antibody binding can be detected by any suitable means, e.g., FACS. The binding consistency, i.e., interpatient variability, can also be evaluated.

### Example 7

### Antibody-Drug Conjugate (ADC) Assays

Antibody-Drug Conjugate (ADC) assays can be carried out on a suitable AML cell lines (e.g., HL60 and OCI AML-5), as well as recombinant 293 cells expressing CLL-1. Briefly, cells are incubated with various concentrations of ADCs for 72-120 hours at 37° C. Cell viability is determined by CellTiter-Glo (Promega) luminescent cell viability assay to determine IC50 values.

### Example 8

### In Vivo Inhibition of AML Tumor Growth

The CLL-1 CYSMAB ADCs can be evaluated for in vivo efficacy. Suitable studies include both (1) subcutaneous (SC) tumor engraftment and growth model utilizing the CLL-1 positive HL60 AML human cell line in mice, and (2) an orthotopic (bone marrow, blood, spleen and lymph node) tumor engraftment and outgrowth model utilizing the CLL-1 positive HL60 or OCI AML-5 human AML cell line.

An established SC HL60 study can be carried out as follows. Animals (nu/nu mice) were inoculated with of 5×10⁶ or 10⁷HL60 cells. Tumor-bearing mice were randomized to a mean tumor volume of 100-150 mm³ in each group (8 animals/group). CLL-1 CYSMAB ADC, or an IgG control ADC, is administered i.p. at a dose of 5-200 μg/animal. Mean tumor volumes were plotted over a time (post-dose).

The OCI AML-5 cell orthotopic studies can be carried out as follows. Immunodeficient NSG mice are split into 5 groups of 8 animals/group. CLL-1 CYSMAB ADC, or an IgG control ADC, are administered i.p. at a dose of 5-200 μg/animal at post (day-6) intravenous inoculation of 5×1⁰⁶ or 1⁰⁷ OCI AML-5 cells. Animals then receive additional antibody doses once per week for the next 2 weeks. The study terminates 4 weeks after administration of the OCI AML-5 cells.

### Example 9

### Specificity for AML Stem Cells in ADC Assays

The specificity of the CLL-1 CYSMAB ADCs prepared according to the disclosure can be tested for specific killing in an ADC assay. Primary patient AML cells or normal CD34 positive hematopoietic stem cells are isolated from the bone marrow of human subjects, and are seeded into a soft agar colony formation assay (100,000 cells/plate). The cells are then incubated in the presence CLL-1 CYSMAB ADCs 14 days. The ADC can cause selective, specific inhibition of AML stem cell clonogenic growth, while normal HSCs should not be affected. The effect of the conjugation can be compared to the naked parent antibody. The negative controls are untreated or treated with an unrelated IgG-ADC.

### Example 10

### Stability of Various Drug Conjugates

A variety of antibody-drug conjugates were made as follows:

| Name | CYSMAB | Concentration | Amount | DAR |
|---|---|---|---|---|
| C6-Cys-02- | S156C | 3.20 mg/ml | 3.1 mg | >1.8 |
| D202 | | | | |
| Cys-01-D202 | A118C | 2.44 mg/ml | 2.8 mg | >1.8 |
| Cys-87-D202 | G316C | 2.63 mg/ml | 2.9 mg | <1.0 |
| Cys-37-D202 | V266C | 4.28 mg/ml | 2.7 mg | >1.8 |
| Cys-10-D202 | S239C | 3.72 mg/ml | 2.6 mg | >1.8 |

All of the conjugates were made with an anti-CLL-1 antibody having the following variable regions: light chain variable region sequence comprising: DIQMTQSPSSLSASVGDRVTLTCRATQELSGYLSWLQQKPGKAIKRLIYAASTLDSGV PSRFSGNRAGTDYTLTISSLQPEDFATYYCLQYAIYPYTFGQGTKLEIK (SEQ ID NO:19) and a heavy chain variable region sequence comprising: EVQLVQSGAEVKKPGASVKMSCKASGYTFTSYFIHWVRQAPGQGLEWIGFINPYND GSKYAQKFQGRATLTSDKSTSTVYMELSSLRSEDTAVYYCTRDDGYYGYAMDYWG QGTLVTVSS (SEQ ID NO:20). The antibodies were conjugated to the indicated cysteine substitution via a cysteine reactive linker to an isoquinolidinobenzodiazepine dimer as follows:

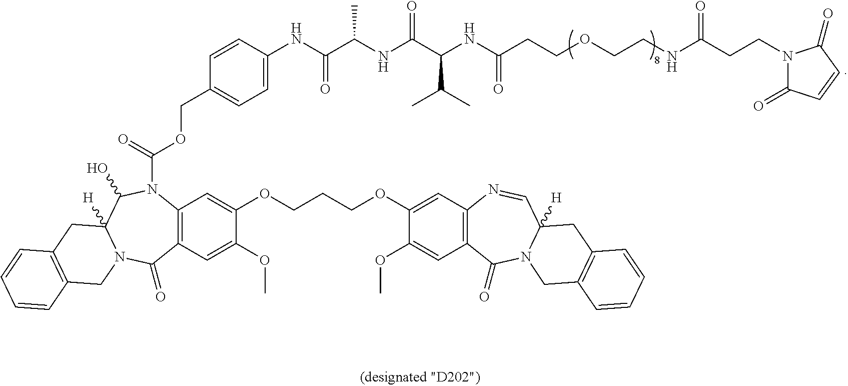

A humanized, cys-substituted anti-CLL1 antibodies in PBS were exchanged into borate buffer (50 mM, pH 8.5, 1 mM diethylene triamine pentaacetic acid (DTPA)) via 2 cycles of molecular weight cut-off filtration (MWCO) using a Millipore, 15 mL, 30 kDa device. To the new solution of the antibodies (5.0 mg/mL, borate buffer (50 mM, pH 8.5, 1 mM DTPA)) was added a solution of Dithiothreitol (DTT) (33 μL, 50.0 equiv., 50 mM) and the resultant solution was shaken gently overnight.

Complete reduction of the interchain disulfide bridges and removal of the substituted cysteine cysteine/glutathione adducts were confirmed by rp-LCMS as described earlier (Junutula et al., 2008, Nature Biotech, 26, 925-932). DTT was then removed from the solution via 3 cycles of molecular weight cut-off filtration (MWCO) using a Millipore, 15 mL, 30 kDa device, using PBS as the exchange buffer. To a 5 mg/ml solution of the fully reduced antibody was added a solution of dehydro ascorbic acid (dhAA) (33 μL, 50.0 equiv., 50 mM). The resultant solution was shaken gently for 3 hrs. The re-oxidation was monitored via rp-LCMS. Once the re-oxidation was deemed complete, the reaction mixture was diluted up to 50% v/v with propylene glycol and D202 was added as a solution in DMSO (10.0 equiv., 10 mM in DMSO).

The reaction was allowed to stir at ambient temperature for 1 hr. The mixture was then treated with activated charcoal for 1 hr at ambient temperature. The activated charcoal was then removed via filtration. The conjugate was then exchanged into PBS via multiple cycles of molecular weight cut-off filtration (MWCO) using Millipore, 15 mL, 30 kDa devices. The solution was then subjected to a sterile filtration to yield the desired conjugate.

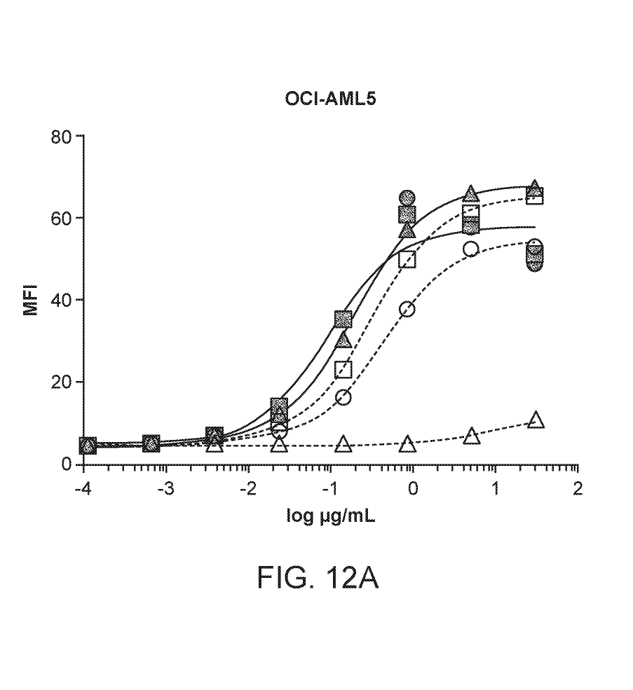

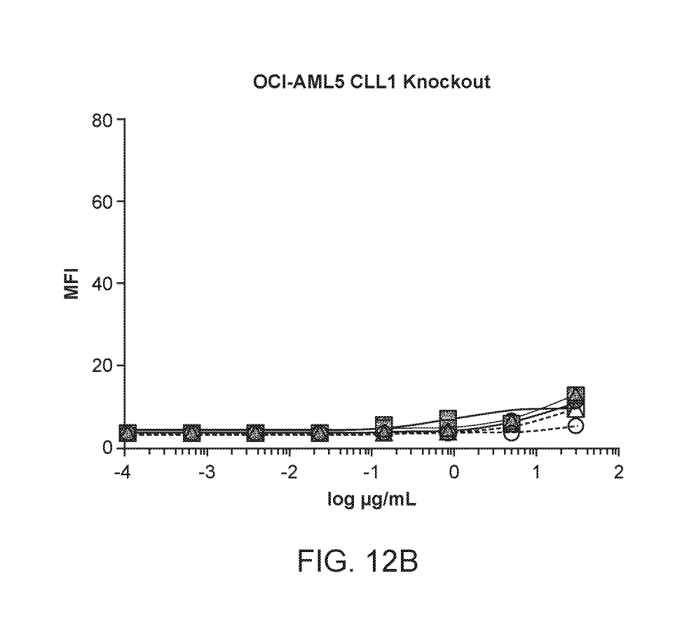

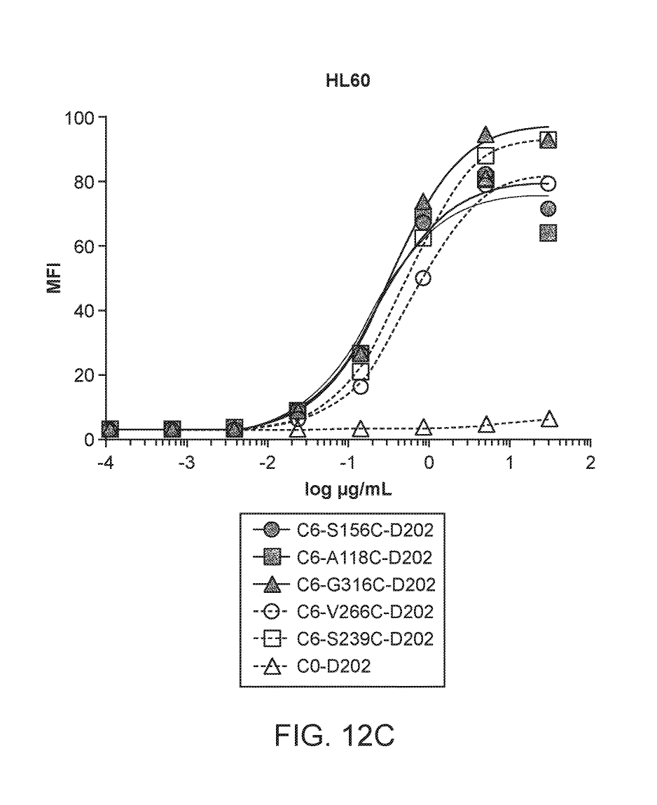

Starting at 30 μg/mL, C6-CYSMAB-D202 ADCs and CO-D202 were subjected to 8-point, 6-fold serial dilutions using cell binding buffer (PBS, with 2% fetal bovine serum). HL60, OCI-AML5 and OCI-AML5-CLL1 knockout cells were washed by staining media and incubated with 5% normal mouse serum on ice for 30 minutes to block Fcγ receptors. The cells were then dispensed into 96-well plate in a density of 0.1e⁶ cells per well and medium was removed by centrifugation. The cell plates were incubated with 100 μL ADC sample dilutions for 30 minutes on ice followed by three times washing and further stained with Alexa-488 conjugated Goat anti human IgG as secondary antibody for 30 minutes on ice. The cells were then washed three times and resuspended in 100 μL cell binding buffer using propidium iodide as cell viability dye. The ADC binding to the cell samples were analyzed by flow cytometry and data analysis by Flowjo. The MFI (Geom. Mean) of FITC signal were plotted using Graphpad Prizm 6. See, FIG. 12A-C.

Stability of the conjugates was determined as follows. On day 0, dilute C6-CYSMAB-D202 ADC samples in human plasma to 200 mg/mL. Perform 9-point, 6-fold serial dilution using human plasma as a diluent. Seal the sample dilution plate and incubate at 37° C. in the CO₂ incubator for 5 days as a sample D5. Prepare Sample D3, D1 and D0 by repeating this plasma dilution and 37° C. incubation procedure on day 2, 4 and 5. On day 5, set up cell killing assay by adding 5 mL of sample D0, D1, D3 and D5 into 95 mL of OCI-AML2 and HL60 cells, incubate at 37° C. for 5 days and quantify the cell viability by Cell-Titer-Glo.

C6-CYSMAB-D202 ADC sample dilutions were carried out in 96-well plates using human plasma as diluent and incubated at 37° C. in the CO₂ incubator for plasma stability study. 9-point, 6-fold serial dilutions, starting at 200 μg/mL ADC, were performed on day 0, 2, 4, and day 5 and the sealed sample dilution plates plus no ADC, plasma only control, were incubated at 37° C., 5% CO₂ incubator for 5, 3, 1 and 0 days, respectively, as sample D5, D3, D1 and D0. In day 5, OCI-AML2 and HL60 cells were seeded in 96-well plates at a density of 2,000 cells in 95 μL Alpha-MEM and IMDM cell culture media, supplemented with 20% fetal bovine serum (FBS) and treated with 5 μL samples from D0, D1, D3, D5 sample dilution plates in triplicates. The assay plates were then incubated at 37° C., 5% CO₂ incubator for 5 days. Live cells (percent of viable cells) were assayed using CellTiter-Glo kit (Promega) and the luminescence measured by a plate reader (Molecular Device Spetramax M5). The results were expressed in percentage of viable cells relative to no ADC plasma only control cells. Individual dose response curves and inhibitory drug concentrations (IC₅₀) were derived by nonlinear regression using Graphpad Prizm 6.

Stability Test of C6-CYSMAB-D202 ADCs on AML2, HL60 Cell Killing is exemplified in the following tables:

AML2

| IC50 μg/mL | D 0 | D 1 | D 3 | D 5 |
|---|---|---|---|---|
| S156C | 0.0016 | 0.0012 | 0.0011 | 0.0058 |
| A118C | 0.0017 | 0.0010 | 0.0028 | 0.0043 |
| G316C | 0.0037 | 0.0019 | 0.0084 | 0.0128 |
| V266C | 0.0041 | 0.0028 | 0.0043 | 0.0070 |
| S239C | 0.0021 | 0.0009 | 0.0008 | 0.0021 |

HL60

| IC50 μg/mL | D 0 | D 1 | D 3 | D 5 |
|---|---|---|---|---|
| S156C | 0.0072 | 0.0070 | 0.0092 | 0.0399 |
| A118C | 0.0100 | 0.0116 | 0.0245 | 0.0608 |
| G316C | 0.0256 | 0.0315 | 0.1153 | 0.1772 |
| V266C | 0.0154 | 0.0115 | 0.0200 | 0.0394 |
| S239C | 0.0087 | 0.0047 | 0.0043 | 0.0120 |

1-70. (canceled)

71. A cysteine-substituted immunoglobulin polypeptide comprising a substituted amino acid residue selected from the group consisting of V266C, G316C, H285C, R301C, V303C, T307C, Y436C, and L441C under EU numbering.

72. The cysteine-substituted immunoglobulin polypeptide of claim 71, wherein the immunoglobulin polypeptide is derived from a human IgG1, IgG2, IgG3 or IgG4 heavy chain constant region.

73. A nucleic acid molecule comprising a nucleotide sequence encoding the cysteine-substituted immunoglobulin polypeptide of claim 71.

74. A recombinant cell comprising the nucleic acid molecule of claim 73.

75. A process for making a cysteine-substituted immunoglobulin polypeptide comprising culturing the recombinant cell of claim 74.

76. A cysteine-substituted antibody comprising the cysteine-substituted immunoglobulin polypeptide of claim 71 in a heavy chain constant region, wherein in the heavy chain constant region is derived from a human IgG isotype selected from the group consisting of IgG1, IgG2, IgG3 and IgG4.

77. The cysteine-substituted antibody of claim 76, further comprising an immunoglobulin light chain selected from the group consisting of kappa and lambda.

78. The cysteine-substituted antibody of claim 76, wherein the antibody binds to CLL-1, GPR114, IL1RAP, TIM-3, CD19, CD20, CD22, ROR1, mesothelin, CD33, CD123/IL3Ra, c-Met, PSMA, prostatic acid phosphatase (PAP), CEA, CA-125, Muc-1, AFP, Glycolipid F77, EGFRvIII, GD-2, NY-ESO-1 TCR, tyrosinase, TRPI/gp75, gp100/pmel-17, Melan-A/MART-1, Her2/neu, WT1, EphA3, telomerase, HPV E6, HPV E7, EBNA1, BAGE, GAGE and MAGE A3 TCRSLITRK6, ENPP3, Nectin-4, CD27, SLC44A4, CAIX, Cripto, CD30, MUC16, GPNMB, BCMA, Trop-2, Tissue Factor (TF), CanAg, EGFR, αv-integrin, CD37, Folate Receptor, CD138, CEACAM5, CD56, CD70, CD74, GCC, 5T4, CD79b, Steap1, Napi2b, Lewis Y Antigen, LIV, c-RET, DLL3, EFNA4, or Endosialin/CD248.

79. The cysteine-substituted antibody of claim 76, comprising a variable light chain and a variable heavy chain, wherein:
(a) the variable light chain comprises a CDR-L1 having the sequence of ESVDSYGNSF (SEQ ID NO:1), CDR-L2 having the sequence of LAS (SEQ ID NO:2), and CDR-L3 having the sequence of QQNNYDPWT (SEQ ID NO:3), and the variable heavy chain comprises a CDR-H1 having the sequence of GYTFTSYV (SEQ ID NO:4), CDR-H2 having the sequence of INPYNDGT (SEQ ID NO:5), and CDR-H3 having the sequence of ARPIYFDNDYFDY (SEQ ID NO:6), or
(b) the variable light chain comprises a CDR-L1 having the sequence of RATQELSGYLS (SEQ ID NO:13), CDR-L2 having the sequence of AASTLDS (SEQ ID NO:14), and CDR-L3 having the sequence of LQYAIYPYT (SEQ ID NO:15), and the variable heavy chain comprises a CDR-H1 having the sequence of GYTFTSYFIH (SEQ ID NO:16), CDR-H2 having the sequence of FINPYNDGSK (SEQ ID NO:17), and CDR-H3 having the sequence of DDGYYGYAMDY (SEQ ID NO:18).

80. A nucleic acid molecule comprising a nucleotide sequence encoding the cysteine-substituted antibody of claim 76 or a portion thereof.

81. A recombinant cell comprising a nucleic acid molecule of claim 80.

82. A process for making an antibody comprising culturing the recombinant cell of claim 81.

83. An antibody conjugate comprising a cysteine-substituted antibody covalently conjugated through a linker to a moiety selected from the group consisting of a drug, a radionucleotide, a fluorophore, a biotin, an RNA, an antibiotic, a protein, and a detectable moiety,
wherein the cysteine-substituted antibody comprises a cysteine-substituted immunoglobulin polypeptide comprising a substituted amino acid residue selected from the group consisting of V266C, G316C, H285C, R301C, V303C, T307C, Y436C, and L441C under EU numbering, and
wherein the cysteine-substituted antibody is covalently conjugated from the substituted amino acid residue in the antibody.

84. A pharmaceutical composition comprising the antibody conjugate of claim 83 and an adjuvant.

85. A method of treating cancer comprising administering to a patient a therapeutically effective amount of the antibody conjugate of claim 83, wherein the antibody conjugate is capable of binding to a tumor associated antigen or cancer stem cell antigen.

86. The method of claim 85, wherein the cancer is a myeloproliferative disorder selected from the group consisting of AML, CML, CMML, multiple myeloma, plasmacytoma, and myelofibrosis.

87. The method of claim 85, wherein the tumor associated antigen or cancer stem cell antigen is CLL-1.

88. A method of detecting a cell expressing CLL-1 comprising:
(a) contacting a cell with an effective amount of a cysteine-substituted antibody of claim 76 capable of binding the cell, and
(b) detecting binding of the cysteine-substituted antibody to the cell,
wherein the binding indicates the cell expressing CLL-1.

89. A method of diagnosing a disease comprising:
(a) contacting a biological sample from an individual with an effective amount of the cysteine-substituted antibody of claim 76 capable of binding to a diseased cell; and
(b) detecting binding of the cysteine-substituted antibody to the disease cell,
wherein the binding indicates the presence of the disease.

90. A method of inhibiting cell division comprising contacting a cell with at least an effective amount of the antibody conjugate of claim 83 capable of binding


SEQUENCE LISTING (20 sequences)

SEQ ID NO: 1 - protein, 10 aa, Homo sapiens
ESVDSYGNSF

SEQ ID NO: 2 - protein, 3 aa, Homo sapiens
LAS

SEQ ID NO: 3 - protein, 9 aa, Homo sapiens
QQNNYDPWT

SEQ ID NO: 4 - protein, 8 aa, Homo sapiens
GYTFTSYV

SEQ ID NO: 5 - protein, 8 aa, Homo sapiens
INPYNDGT

SEQ ID NO: 6 - protein, 13 aa, Homo sapiens
ARPIYFDNDYFDY

SEQ ID NO: 7 - protein, 111 aa, Homo sapiens
TIVLTQSPASLAVSLGQRATISCRASESVDSYGNSFMHWYQQKPGQPPKLLIYLASNLESGVPARFSGSGSRTDFTLTIDPVEADDAATYYCQQNNYDPWTFGGGTKLEIK

SEQ ID NO: 8 - protein, 111 aa, Homo sapiens
DIVMTQSPDSLAVSLGERATINCRASESVDSYGNSFMHWYQQKPGQPPKLLIYLASNLESGVPDRFSGSGSGTDFTLTISSLQAEDVAVYYCQQNNYDPWTFGGGTKVEIK

SEQ ID NO: 9 - protein, 107 aa, Homo sapiens [sequence omitted]

SEQ ID NO: 10 - protein, 104 aa, Homo sapiens
PKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 11 - protein, 120 aa, Homo sapiens
EVQLQQSGPELVKPGASVKMSCKASGYTFTSYVMHWVKQKPGQGLEWIGYINPYNDGTKYNEKFKGKATLTSDTSSSTAYMELNSLTSEDSAVYFCARPIYFDNDYFDYWGQGTTLKVSS

SEQ ID NO: 12 - protein, 120 aa, Homo sapiens
QVQLVQSGAEVKKPGASVKVSCKASGYTFTSYVMHWVRQAPGQRLEWIGYINPYNDGTKYNEKFKGKATITSDTSASTAYMELSSLRSEDTAVYYCARPIYFDNDYFDYWGQGTLVTVSS

SEQ ID NO: 13 - protein, 11 aa, Homo sapiens
RATQELSGYLS

SEQ ID NO: 14 - protein, 7 aa, Homo sapiens
AASTLDS

SEQ ID NO: 15 - protein, 9 aa, Homo sapiens
LQYAIYPYT

SEQ ID NO: 16 - protein, 10 aa, Homo sapiens
GYTFTSYFIH

SEQ ID NO: 17 - protein, 10 aa, Homo sapiens
FINPYNDGSK

SEQ ID NO: 18 - protein, 11 aa, Homo sapiens
DDGYYGYAMDY

SEQ ID NO: 19 - protein, 107 aa, Homo sapiens
DIQMTQSPSSLSASVGDRVTLTCRATQELSGYLSWLQQKPGKAIKRLIYAASTLDSGVPSRFSGNRAGTDYTLTISSLQPEDFATYYCLQYAIYPYTFGQGTKLEIK

SEQ ID NO: 20 - protein, 120 aa, Homo sapiens
EVQLVQSGAEVKKPGASVKMSCKASGYTFTSYFIHWVRQAPGQGLEWIGFINPYNDGSKYAQKFQGRATLTSDKSTSTVYMELSSLRSEDTAVYYCTRDDGYYGYAMDYWGQGTLVTVSS